(19) United States
(12) Patent Application Publication
(10) Pub. No.: US 2020/0246364 A1
(43) Pub. Date: Aug. 6, 2020

(54) MEDICAL APPLICATION OF COMPOSITION OF FRUCTOSE-1,6-BISPHOSPHATE AND BLOOD CONCENTRATION STABILIZER THEREOF

(71) Applicant: ZHEJIANG UNIVERSITY, Hangzhou City, Zhejiang Province (CN)

(72) Inventors: Xiaoyuan LIAN, Hangzhou City, Zhejiang Province (CN); Zhizhen ZHANG, Hangzhou City, Zhejiang Province (CN); Heng QIAN, Hangzhou City, Zhejiang Province (CN); Hongyun BI, Hangzhou City, Zhejiang Province (CN); Binbin CHI, Hangzhou City, Zhejiang Province (CN); Rongyao ZHU, Hangzhou City, Zhejiang Province (CN)

(21) Appl. No.: 16/637,766

(22) PCT Filed: Dec. 4, 2017
(86) PCT No.: PCT/CN2017/114371

§ 371 Date: Feb. 10, 2020

(30) Foreign Application Priority Data
Aug. 10, 2017 (CN) 201710678435.5

(51) Int. Cl. A61K 31/7024; A61K 45/06; A61K 31/4985; A61P 3/00

(52) U.S. Cl. CPC A61K 31/7024; A61P 3/00; A61K 31/4985; A61K 45/06

(57) ABSTRACT

Application of a composition of FBP and a blood concentration stabilizer thereof in manufacturing medicaments for preventing and treating metabolic diseases and metabolic dysfunction related diseases. The FBP may also be a pharmaceutically acceptable salt or hydrate of prototype thereof, a prodrug thereof, or a derivative thereof. The blood concentration stabilizer refers to a medicament or substance for treating diabetes capable of slowing down rapid in vivo degradation of FBP in a pharmaceutical preparation. The composition can cause a higher FBP blood concentration peak value and a more stable blood concentration, and can reduce an FBP dosage and thus can reduce the toxicity resulted from a large amount of inorganic phosphorus entering systemic circulation after degradation of a large dosage of FBP.

### TECHNICAL FIELD

The present disclosure relates to the pharmaceutical field, and particularly to uses of a composition of fructose-1,6-bisphosphate (also known as fructose-1,6-diphosphate and fructose disphosphate) and a blood concentration stabilizer thereof in manufacturing medicaments for preventing and treating metabolic diseases and metabolic dysfunction related diseases, and the diseases including tumors, fatty liver, diabetes, hyperlipidemia, cardiovascular diseases, peripheral neurological diseases, and central nervous diseases.

### BACKGROUND

Fructose-1,6-bisphosphate (FBP) is an intermediate of glycometabolism present in the body. Exogenous FBP produces pharmacological action by regulating activities of several enzymes involved in glycometabolism (instructions for fructose diphosphate sodium tablets, instructions for the second batch of chemicals released by the State Drug Administration in 2002). Exogenous FBP has a variety of pharmacological effects such as increasing concentrations of intracellular adenosine triphosphate and phosphocreatine, promoting potassium influx, increasing a content of diphosphoglyceric acid in red blood cells, and inhibiting release of oxygen free radicals and histamine, and it also can reduce body damages caused by ischemia and hypoxia, and particularly has good protective effect on the ischemic heart disease. In China, a variety of FBP preparations have been put in clinical use, such as for adjuvant therapies of shock, coronary atherosclerotic heart disease (coronary heart disease), angina pectoris, acute myocardial infarction, heart failure and arrhythmia (instructions for fructose diphosphate sodium injection, instructions for fructose diphosphate sodium tablets, and instructions for the second batch of chemicals released by the State Drug Administration in 2002). FBP has the effects of treating diabetes, or diabetic and combined cardio-cerebrovascular diseases (Chinese invention patent: CN00112023.9). The applicant also disclosed the anti-epileptic effect of FBP (Chinese invention patent: ZL201310498212.2) and the antitumor effect of FBP (Chinese invention patent: ZL201110066413.6). In particular, the anti-epileptic effect of FBP is significantly better than the existing anti-epileptic medicaments with respect to the function of repairing epileptic brain and controlling the onset of epilepsy at the same time, and it also can significantly improve the cognitive ability of epileptic animals and has sustainable antiepileptic effect after drug withdrawal, and has a broad-spectrum and remarkable anti-cancer effect while being highly safe for normal cells. In view of the above, FBP has great potential medicinal values.

However, the medicinal values of existing FBP preparations are limited by the in vivo metabolic characteristics of the exogenous FBP. The existing fructose-1,6-bisphosphate preparations are administrated with great doses (recommended oral preparation: 1 g each time, 3-4 times per day; intravenous treatment: 10 g per day, in 2 intravenous infusions). Studies have shown that the currently recommended oral doses fail to significantly increase FBP levels in the blood, and thus it is recommended to increase the clinical administration dose (Acta Pharm. 65 (2015) 147-157). The exogenous FBP can be rapidly degraded in vivo. After healthy volunteers accept an intravenous infusion of this product (250 mg/kg), a blood concentration thereof can reach 770 mg/L within 5 minutes, and a half-life is about 10-15 minutes. The product is eliminated from the plasma by hydrolysis into inorganic phosphorus and fructose, and only a small part thereof is excreted from the urine (instructions for fructose diphosphate sodium injection, and instructions for the second batch of chemicals released by the State Drug Administration in 2002). The research published by the applicant further substantiates that the blood concentration of FBP gradually decreases with the treatment time; and this phenomenon becomes more serious with an increase in the dose (Chinese invention patent: ZL201310498212.2). Consistently, FBP has antiepileptic and anticancer effects in a small dose range, and specifically, a long-term effective dose for chronic epilepsy in rats is in a range of 100-200 mg/kg/day administered intragastrically, and an effective dose for mouse tumor models is in a range of 400-450 mg/kg/time injected intraperitoneally. It can be seen that the blood concentration of FBP cannot be increased by simply increasing the dose of FBP, which limits the medicinal applications of FBP. Therefore, it is of great significance for the extensive medicinal applications of FBP to verify the in vivo metabolic mechanism of the exogenous FBP and search for methods and substances for stabilizing the blood concentration of the exogenous FBP.

The wide range of pharmacological activities of FBP cannot be explained by the current knowledge of the mechanism of FBP. It is uncertain whether there is a common biological activity that supports the extensive pharmacological activities of FBP and thus supports a wide range of clinical therapeutic effects. More and more studies have proved that the metabolic disorder or dysfunction is a common pathological mechanism of many diseases. These diseases include various severe diseases such as diabetes and its complications, cardiovascular disease, neurological disorders (epilepsy, schizophrenia, depression, etc.), neurodegenerative diseases (senile dementia, vascular dementia, Parkinson's disease, multiple sclerosis, etc.), tumors, etc. Among them, the senile dementia is now also known as type 3 diabetes (Biochem Pharmacol. 2014 Apr. 15; 88(4):548-59. Eur Neuropsychopharmacol. 2014 December; 24(12):1954-60. Neurol Sci. 2015 October; 36(10):1763-9.), and tumors were called as metabolic diseases by Otto Warburg, a German biochemist and Nobel Prize winner, in the 1960s. Mitochondrial dysfunction or abnormality is a common metabolic feature of the aforementioned diseases. For example, the mitochondrial dysfunction occurs in various nerve pains caused by different reasons, including chemotherapy-induced neuropathy, diabetic neuropathy, and traumatic neuropathy (Mol Pain. 2015; 11:58.), and malignant chain reactions induced by the mitochondrial dysfunction include energy substances (ATP) insufficiency caused by functional weakening of oxidative phosphorylation, oxidative stress injury caused by an increased production and a reduced elimination of reactive oxygen species (ROS), and inflammatory response, which is a common pathological mechanism of nerve pains induced by anticancer drugs and other reasons (Pain. 2013 November; 154(11):2432-40. Neurosci Lett. 2015 Jun. 2; 596:90-107. Curr Neuropharmacol. 2016; 14(6):593-609.), which is also a common pathological event of other diseases mentioned above (Nature. 2006 Oct. 19; 443(7113):787-95; Neurobiol Dis. 2013 March;51:27-34; Biomed Pharmacother. 2015 August;74:101-10. Biochim Biophys Acta. 2017 May; 1863(5):1037-1045; Biochim Biophys Acta. 2017 May; 1863(5):1132-1146). In particular, the researches over the past ten years have revealed more features of tumor metabolism, and confirmed that tumor metabolic reprogramming is a core feature of cancer and is closely related to occurrence and progression of tumor as well as drug resistance in cancer treatment. Through the metabolic reprogramming, cancer cells can use usual nutrients, especially glucose and glutamine, to simultaneously meet energy requirements, redox balance, and highly active biosynthesis, thereby ensuring the prerequisites for rapid division and immortalization of tumor cells. Tumor epigenetic abnormalities are closely related to anti-cancer gene quiescence and cancer promoting gene over-expression. Recent studies have demonstrated that tumor characteristic metabolism also maintains epigenetic characteristics of tumors. Therefore, regulating metabolism and/or reversing the pathological metabolic mode back to the normal metabolic mode may have broad application prospects for the prevention and treatment of the above metabolic diseases and metabolism-related diseases. Fructose-1,6-bisphosphate, as an intermediate of glycolysis metabolism and gluconeogenesis, may have extensive metabolic regulation effect and/or effect of reversing the pathological metabolic pattern to the normal metabolic pattern, and thus may have a wide range of medicinal applications for preventing and treating metabolic diseases and metabolism-related diseases. Apparently, it is conducive to utilizing the medicinal value thereof to systematically reveal the regulation of exogenous fructose-1,6-bisphosphate (FBP) on cell metabolism including tumor cell metabolism and its mechanism.

### SUMMARY

An object of the present disclosure is to provide a pharmaceutical use of a composition of the fructose-1,6-bisphosphate and a blood concentration stabilizer thereof, which is a use of a composition consisting of a fructose-1,6-bisphosphate and a blood concentration stabilizer in manufacturing a medicament for preventing and treating metabolic diseases and metabolic dysfunction related diseases, and also a use of a composition consisting of a fructose-1,6-bisphosphate (FBP) and a substance capable of stabilizing an FBP blood concentration (generally referred to as FBP blood concentration stabilizer) in manufacturing a medicament for preventing and treating the metabolic diseases and metabolic dysfunction related diseases. The medicament includes a therapeutically effective amount of fructose-1,6-bisphosphate, an effective dose of a blood concentration stabilizer, and a pharmaceutically acceptable excipient or carrier. A ratio of FBP to stabilizer in the medicament is determined on the premise that the stabilizer can exert its effect of stabilizing the FBP blood concentration, and thus different stabilizers may have different ratios to FBP. When the medicament is administrated to prevent and treat the metabolic diseases and metabolic dysfunction related diseases, an effective dose thereof is determined by the specific disease, and for example, a dose of FBP for the treatment of tumor is 1 to 5 times higher than that for the treatment of other diseases.

Pharmaceutical forms of FBP include prototype fructose-1,6-bisphosphate, and pharmaceutically acceptable salts of fructose-1,6-bisphosphate and prodrugs or derivatives thereof, including, but not limited to, salts and hydrates formed by ammonium, sodium, potassium, calcium, magnesium, manganese, copper, methylamine, dimethylamine, trimethylamine, butyric acid, acetic acid, dichloroacetic acid, hydrochloric acid, hydrobromic acid, sulfuric acid, trifluoroacetic acid, citric acid or acid radical of maleic acid that forms the compound. Preferably, the pharmaceutical form is an 8-molecule hydrate of trisodium fructose-1,6-bisphosphate.

The blood concentration stabilizer refers to a diabetes medicament or substance capable of slowing a rapid in vivo degradation of fructose-1,6-bisphosphate in the medicament preparation, including dipeptidyl peptidase-4 (DPP-4) inhibitors such as sitagliptin, glucagon-like peptide 1 (GLP-1) receptor agonists, biguanides such as metformin, insulin and glitazones also known as thiazolidinediones, and fructose-1,6-bisphosphatase inhibitors. In a composition consisting of the fructose-1,6-bisphosphate and any of the stabilizers, a ratio of the fructose-1,6-bisphosphate to the stabilizer is as follows: a ratio of the 8-molecule hydrate of trisodium fructose-1,6-bisphosphate (gram) to metformin (gram) is 1:0.1 to 1:1, preferably 1:0.2 to 1:1; a ratio of the 8-molecule hydrate of trisodium fructose-1,6-bisphosphate (gram) to sitagliptin (gram) is 1:0.001 to 1:0.5, preferably 1:0.01 to 1:0.1; and a ratio of the 8-molecule hydrate of trisodium fructose-1,6-bisphosphate (gram) to insulin (unit: IU) is 1:0.02 to 1:0.002, preferably 1:0.006 to 1:0.008.

The metabolic diseases and metabolic dysfunction-related diseases specifically include existing indications of the fructose-1,6-bisphosphate preparations (mainly including: adjuvant treatments for improving myocardial ischemia and viral myocarditis caused by angina pectoris of coronary heart disease, acute myocardial infarction, arrhythmia, and heart failure); cerebral infarction; cerebral hypoxia caused by cerebral hemorrhage or the like; blood system cancer, various solid tumors; diabetes and complications; fatty liver; epilepsy; neurodegenerative diseases (including senile dementia, Parkinson's disease, multiple sclerosis); and psychobehavioral disorders.

The present disclosure reveals the extensive regulating effects of exogenous fructose-1,6-bisphosphate (FBP) on metabolic activities, and particularly, the protective regulating effects on normal cells and the characteristics of reversing tumor metabolism, which provides a scientific basis for that FBP can protect the normal cells and also functions to eliminate a variety of cancer cells, which also provides support for the medicinal use of FBP in the prevention and treatment of various metabolic diseases and diseases relating to metabolic dysfunctions or disorders. These diseases include diabetes and its complications, cardiovascular disease, neurological disorders (epilepsy, schizophrenia, depression, etc.) and neurodegenerative diseases (senile dementia, vascular dementia, Parkinson's disease, multiple sclerosis, etc.), and tumors. More importantly, the present disclosure reveals the mechanism by which exogenous FBP is rapidly degraded and provides a group of substances (hereinafter referred to as FBP blood concentration stabilizer) that can slow the in vivo degradation rate of FBP, in turn increase the peak value of FBP blood concentration and extend its half-life period, and significantly enhance the efficacy of FBP. This supports the medicinal use of a combination of FBP and its blood concentration stabilizer, i.e., a composition of FBP and its blood concentration stabilizer, in manufacturing a new FBP medicament with FBP as an active pharmaceutical component. Therefore, the present disclosure not only provides a group of novel FBP medicaments that can prevent FBP from being rapidly degraded in vivo, but also expands the medical application range of FBP.

The novel FBP medicament includes FBP and a FBP blood concentration stabilizer for inhibiting the rapid degradation of FBP in vivo. The FBP blood concentration stabilizer can slow down an acute degradation of FBP in vivo and block an accelerated degradation with the treatment time. In this way, the pharmaceutical preparation of this composition can produce a higher peak of FBP blood concentration (after treatment one time or multiple times) and prolong its half-life period. Therefore, the preparation of the composition can not only enhance various pharmacological effects of FBP and expand the dose range of FBP, but also can alleviate a phosphoric acid poisoning phenomenon of the existing FBP preparation caused by rapid increase of phosphoric acid level due to the rapid degradation of FBP. In particular, this preparation of FBP composition is effective for long-term administration, and thus the medicinal values of FBP in the treatment of chronic diseases including tumors, epilepsy, diabetes, and neurodegenerative diseases can be fully exerted. In summary, the FBP blood concentration stabilizer remedies the defects that FBP itself as a metabolic intermediate is rapidly degraded in vivo, and thus provides a breakthrough improvement of the medicinal values of FBP.

The FBP blood concentration stabilizer refers to active substances capable of slowing the in vivo degradation of exogenous FBP, including existing hypoglycemic agents and emerging hypoglycemic agents that will be continuously developed in the future, as well as newly found or orientation-synthesized substances capable of indirectly or directly inhibiting fructose-1,6-bisphosphatase (FBPase). The existing hypoglycemic agents include dipeptidyl peptidase-4 (DPP-4) inhibitors such as sitagliptin, glucagon-like peptide 1 (GLP-1) receptor agonists, biguanides such as metformin, insulin, glitazones (also known as thiazolidinediones), and fructose-1,6-bisphosphatase inhibitors (such as fructose-2,6-bisphosphate). In practical applications, FBP and one or more of the above FBP blood concentration stabilizers may, in an appropriate ratio, constitute active components of the medicament.

The exogenous fructose-1,6-bisphosphate (FBP) may extensively regulate cell metabolic activities after entering the body, so as to prevent various metabolic disorders and abnormal metabolism-related diseases. At the same time, after entering the body, the exogenous FBP, as an intermediate of glycometabolism, acts as an energy metabolism substrate which passes through the glycolysis pathway, then enters the tricarboxylic acid circulation, and eventually is oxidatively phosphorylated to produce energy (ATP), and which may also be dephosphorylated to produce glucose and a final product glycogen through an FBPase-initiated gluconeogenesis pathway. That is, these two possible metabolic degradations of FBP can directly lead to a rapid consumption of exogenous FBP after entering the body, and thus it is difficult to produce a sufficient FBP blood concentration for exerting the pharmacological effects and maintain a sufficiently long half-life period. In this way, the exogenous FBP cannot exert its pharmacological activity of regulating metabolism and the corresponding pharmacological effects.

The present application verifies the above scientific hypothesis and finds a solution to the problem.

First, the present disclosure has found that exogenous FBP is not consumed as a substrate for energy metabolism in a cell culture system but exerts the extensive regulating effect on metabolism. In particular, FBP exhibits different metabolic regulating effects on normal cells and tumor cells, and thus provides a scientific basis for that FBP can protect normal cells and their functions as well as eliminate various different cancer cells. Specifically, 1) regardless of cell type, FBP can: (1) promote mitochondrial oxidative phosphorylation activity, thereby increasing the ATP level; 2) promote pentose phosphate metabolic bypass (PPP) of normal cells, and increase levels of endogenous antioxidant NADPH and reduced glutathione (GSH), thereby preventing an oxidative damage on normal cells. In contrast, FBP inhibits PPP in cancer cells, decreases the levels of NADPH and GSH, increases a level of ROS, leads to mitochondrial damage, and induces cancer cell senescence and apoptosis. In addition, FBP can down-regulate multiple key metabolic enzymes in the tumor metabolic network, block the glycolysis intermediates and tricarboxylic acid circulation intermediates flowing to biosynthesis, and reverse the epigenetic characteristics of the tumor.

Then, the present disclosure has found that the protein level of FBPase in the body is significantly increased by repeatedly administrating the exogenous FBP to the whole tumor model animal for a few days, and a higher dose of FBP leads to an earlier occurrence of the up-regulation of FBPase accompanied by a corresponding decrease of FBP blood concentration. These research results not only reveal the scientific fact that FBP has a narrow effective dose range, and further indicate from the mechanism that it is impossible to increase the blood concentration of FBP by increasing the dose of FBP. Based on the key role of FBPase in the rapid in vivo dephosphorylation of FBP, it is reasonable to assume that the up-regulation of FBPase accelerates the rapid in vivo degradation of the exogenous FBP and leads to a gradual decrease in FBP efficacy with the extension of the treatment time, and when the dose of FBP reaches to a certain level, a higher dose may result in worse effect. It is obvious that the key to overcome the rapid in vivo degradation of exogenous FBP is to increase the blood concentration of FBP and prolong its half-life period, which is also pivotal to sufficient exertion of the efficacy of the exogenous FBP.

The above findings indicate that inhibition of the gluconeogenesis pathway of FBP is essential for maintaining the blood concentration of exogenous FBP, and also provide clues and molecular targets for stabilizing the blood concentration of exogenous FBP, especially the blood concentration of FBP for long-term treatment. Theoretically, different types of hypoglycemic agents may inhibit different sections of the gluconeogenesis pathway through different mechanisms, so as to indirectly or directly inhibit the gluconeogenesis of FBP, thereby preventing the rapid in vivo degradation of the exogenous FBP and suppressing a chronic activation of the gluconeogenesis pathway induced by repeated treatments of FBP. In this way, the peak blood concentration and the half-life period of the exogenous FBP is increased, thereby significantly improving the efficacy of FBP preparations.

Therefore, the applicants have studied the stabilizing effects of different types of hypoglycemic agents on the blood concentration of FBP, in order to overcome the defects that FBP is rapidly degraded in vivo and it is unconducive to the exertion of the anticancer efficacy and other efficacies of FBP. The present disclosure has found that, by administrating sitagliptin phosphate, metformin or insulin in clinical dose 0.5 h before the intragastric administration of FBP, the peak blood concentration of FBP after single or multiple intragastric administrations of FBP can be increased, the half-life period of FBP in the blood can be prolonged, and both the up-regulation of the FBPase protein level induced by repeated administration of FBP and the corresponding down-regulation of the blood concentration of FBP can be blocked, thereby significantly enhancing the overall anticancer efficacy of FBP.

Metformin is a classic agent for the treatment of type 2 diabetes and can inhibit excessive gluconeogenesis of liver and kidney, because the dephosphorylation of fructose-1,6-bisphosphate under the catalysis of FBPase is a rate limiting process during the gluconeogenesis. Therefore, the inhibition of the gluconeogenesis by metformin can indirectly inhibit dephosphorylation of the fructose-1,6-bisphosphate as a substance of the gluconeogenesis, thereby increasing the peak blood concentration of exogenous FBP and prolonging the half-life period of FBP. The inhibitory effect of sitagliptin on gluconeogenesis can also indirectly or directly protect FBP from being degraded by dephosphorylation by FBPase. Sitagliptin exerts a hypoglycemic effect by inhibiting dipeptidyl peptidase-4 (DPP-4). The glucagon-like peptide-1 (GLP-1) in the body can play a role in lowering blood glucose through a variety of mechanisms, one of which is to reduce gluconeogenesis. The activity of GLP-1 is negatively regulated by DPP-4, and thus the inhibition of DPP-4 by sitagliptin restores the activity of GLP-1, so as to inhibit the gluconeogenesis and an upstream gluconeogenesis pathway. Similarly, insulin has a hypoglycemic effect by inhibiting glycogen synthesis, and can also protect FBP from being degraded by dephosphorylation. Glitazones, such as troglitazone, can directly inhibit FBPase to increase the peak blood concentration of the exogenous FBP and prolong its half-life period, thereby improving the clinical values of the preparations using FBP as the pharmacologically active component. Fructose-2,6-bisphosphate is an isomer of fructose-1,6-bisphosphate, and is the most active endogenous FBPase inhibitor known so far. FBPs can be combined to prepare a composite preparation having a higher bioavailability of FBP.

It should be noted that many studies here and abroad have reported the antitumor activity of metformin, and the anticancer activity of metformin has attracted extensive attention in the anti-tumor field in the world. However, animal experiments have demonstrated that the effective antitumor dose of metformin is much higher than the dose required for the treatment of diabetes, i.e., the anticancer effect is not achieved by regulating blood glucose level. The present disclosure also has found that, when FBP is combined with a clinical hypoglycemic dose of metformin, the efficacy of FBP in inhibiting tumor growth is enhanced. On the contrary, when combining with a higher dose of metformin which itself has a certain tumor growth inhibiting effect, the anticancer effect of FBP is attenuated. This indicates that, in the present disclosure, FBP is combined with the hypoglycemic dose of metformin, in order to utilize the effect of metformin on stabilizing FBP blood concentration rather than the anticancer effect thereof.

The present invention also found that no significant anticancer activity was observed in the cell culture system of sitagliptin; and on the animal tumor models, the clinical hypoglycemic dose of sitagliptin showed certain anticancer activity in some models. This in vivo anticancer activity may be the result of the regulation of glycometabolism, or the result of other actions such as improving the body's immunity. In particular, the combination of FBP and the hypoglycemic dose of sitagliptin produces stronger anticancer effects than respective separate treatments, which strongly supports the application values of the combination of FBP and sitagliptin in the preparation of novel anticancer medications.

The present invention also found that the combination of FBP and sitagliptin can also significantly inhibit the body weight gain caused by high-fat diet, and such an effect has not been observed in the respective separate treatments; FBP can reduce fat accumulation caused by the high-fat diet, and such an efficacy is enhanced by the combination of FBP and sitagliptin, while the administration of sitagliptin alone does not have such an efficacy. The results of the study not only prove that FBP can promote fat metabolism, but also support the application value of the novel FBP medicament according to the present disclosure in weight loss as well as prevention and treatment of diabetes, especially the type 2 diabetes.

The present invention also found that FBP can significantly resist peripheral neuralgia caused by cancer chemotherapeutic agents, which further supports the application value of the novel FBP medicament according to the present invention in the treatment of cancer. In particular, traditional chemotherapeutic medicaments are still the mainstream anticancer agents in clinical practice, but their poisonous side effects include peripheral neuralgia, which not only seriously affects the quality of life of patients, but often leads patients to abandon chemotherapy. Therefore, it is of great significance for cancer treatment to develop a medicament that can reduce the poisonous side effects of chemotherapy agents without reducing anticancer effect thereof. Thus, FBP anticancer preparations produced according to the present disclosure can also be used in combination with the traditional chemotherapeutic agents in the clinical practice, thereby further enhancing the anticancer effect while overcoming the neurotoxic side reaction. Metabolic dysfunction, especially diminished function of mitochondrial oxidative phosphorylation and malignant chain reactions caused therefrom, including insufficient energy substance ATP, increased ROS production, reduction of endogenous antioxidants, and inflammatory reactions, are the common pathological mechanism of the peripheral neuralgia caused by the anticancer medicaments and nerve pains induced by other reasons. The pharmacological activity of FBP against peripheral neuralgia caused by the anticancer chemotherapeutic medicaments is highly consistent with its function in regulating normal cell metabolism, which also supports the medical use of FBP in the prevention and treatment of nerve pains caused by other reasons.

Regarding the novel FBP medicament according to the present disclosure, the key point thereof is to combine with the blood concentration stabilizer thereof, so as to obtain a higher peak blood concentration of FBP and a longer half-life period, thereby better exerting the efficacy of FBP. Therefore, those skilled in the art will understand that the novel FBP medicament according to the present disclosure is also applicable to the various known indications of FBP, including: adjuvant treatment of myocardial ischemia and viral myocarditis caused by angina pectoris of coronary heart disease, acute myocardial infarction, arrhythmia, and heart failure; improvement for cerebral hypoxia symptoms caused by cerebral infarction, cerebral hemorrhage, etc.; prevention and treatment of blood system cancer and various solid cancers; prevention and treatment of diabetes and its complications, epilepsy and neurodegenerative diseases (including senile dementia, Parkinson's disease, multiple sclerosis).

In the novel FBP medicament prepared by the composition, the medicinal dosage of the 8-molecule hydrate of trisodium fructose-1,6-bisphosphate is 100-5000 mg/kg (body weight/day), preferably 300-2000 mg/kg (body weight/day); the medicinal dosage of metformin is 1-1000 mg/kg (body weight/day), preferably 50-300 mg/kg (body weight/day); the medicinal dosage of sitagliptin is 0.1-500 mg/kg (body weight/day), preferably 1-100 mg/kg (body weight/day); and the medicinal dosage of insulin, depending on the types thereof, varies from 10 to 100 U/kg (body weight/day). The treatment with the novel FBP can be a single treatment or multiple treatments, where the multiple treatments means 2-4 times per day.

The “medicinal dosage” means a dosage that can achieve the purpose of preventing, effectively controlling or treating a disease. In clinical use, the doctors may follow the principle of individualized treatment, and adjusts the dosage of the medicament of the individual according to the patient's disease condition. In this regard, the dosages and ratios of the compositions provided in the present disclosure are not intended to limit the dosages and ratios of the pharmaceutical compositions of the present invention, but are preferred dosages and ratios in the present disclosure.

In the present disclosure, the “patients” are especially human beings. However, it should be understood that within the interpretation scope of the existing pharmacology, the medicinal dosage and range for human can be converted to an appropriate dosage and range for animals, especially mammals such as rat, mouse, dog, etc.

Dosage forms of the novel FBP medicament prepared by the composition include an injection, a common tablet, a granule, a capsule, a double-layer tablet, a controlled release double-layer tablet, a sustained release tablet, a single-chamber controlled release tablet, a dispersible tablet, an enteric-coated tablet, an enteric-coated capsule, a fixed point release tablet, a controlled-sustained release capsule, a sustained release pellet, a capsule containing pellets or small tablets, and a targeting preparation, but are not limited thereto. The preferred dosage form is a controlled release solid preparation which can release the stabilizer first for 15 minutes to 60 minutes and then release FBP; it is also possible that the stabilizer is prepared into an A oral or injection preparation, FBP is prepared into a B oral or injection preparation, and during the clinical use, the A preparation is first used for 15 minutes to 60 minutes, and then the B preparation is used.

The excipient used in the double-layer tablet is selected from, but not limited to, the following excipients: methyl cellulose, hydroxyethyl cellulose, hydroxyethyl methyl cellulose, hydroxypropyl cellulose, hydroxypropyl methyl cellulose, hypromellose, hydroxymethyl cellulose, sodium hydroxymethyl cellulose, glucose, chitin, chitosan, galactomannan, beeswax, hydrogenated vegetable oil, synthetic wax, butyl stearate, stearic acid, carnauba wax, glyceryl stearate, propylene glycol stearate, stearyl alcohol, polyvinyl alcohol and carbopo 934. The stabilizer is selected from sodium citrate or citric acid. The lubricant is selected from magnesium stearate, stearic acid, colloidal silica, or talc.

The preparation method includes direct compression, wet granulation and compression, dry granulation and compression, double compression, and the like.

The wet granulation and compression method is preferred, which has a simple process, is time-saving, and can protect the stability of the medicament. The specific preparation method includes the following steps: respectively mixing active ingredients with a filler and a binder according to a recipe of the layer A and a recipe of the layer B; drying and modifying granules formed after wet granulation; respectively mixing the dried granules of the layer A and the dried granules of the layer B with a disintegrant and a lubricant, and then pressing to obtain a double-layer tablet of the 8-molecule hydrate of trisodium fructose-1,6-bisphosphate and sitagliptin.

In order to improve clinical administration compliance and reduce the times for administration, the composition is prepared into a sustained release pellet containing 8-molecule hydrate of trisodium fructose-1,6-bisphosphate and sitagliptin. The sustained-release pellet is composed of three parts: a blank pellet, a quick-release layer and a sustained-release layer. According to the required sustained release dosage forms, fructose-1,6-bisphosphate is prepared into a coated sustained-release pellet, and the sitagliptin, as a common thin-film coating component, is coated on the outer layer of the sustained-release pellet of fructose-1,6-bisphosphate.

The excipient used in the sustained release pellet is selected from, but not limited to, the following excipients:

1. The blank pellet: the filler is selected from lactose, starch, microcrystalline cellulose, etc.; the binder is selected from sucrose, methylcellulose, hydroxypropylmethylcellulose, polyvinylpyrrolidone, etc.; and the lubricant is magnesium stearate, stearic acid, colloidal silica, talc, etc.

2. The quick-release pellet: a polymer film coating material is polyvinylpyrrolidone, hypromellose, polyethylene glycol, etc.

3. The sustained release pellet: the retarder is acrylic resin, ethyl cellulose, or Chinese insect wax; the porogen is lactose, hypromellose, polyvinylpyrrolidone, or talc; the plasticizer is triethyl citrate, diethyl phthalate, polyethylene glycol 6000, tributyl citrate, dibutyl succinate; and the anticaking agent is talc, magnesium stearate, glyceryl monostearate.

4. The preparation method includes: preparing fructose-1,6-bisphosphate blank pellets by an extrusion-spheronization method, then coating in a fluidized bed, and then directly coating the metformin as a thin-film coating component on outer layer of the fructose-1,6-bisphosphate sustained-release pellets.

Preferably, the specific preparation method is as follows:

(1) Preparation of the blank pellets: weighing the medicament and the excipient, mixing with the sieved excipient, adding water to prepare a soft material, and then obtaining the fructose-1,6-bisphosphate pellets by extrusion-spheronization. The obtained pellets are dried and sieved for subsequent use.

(2) Coating of sustained-release pellet: preparing a coating solution with Eudragit Ne30d (polymer concentration: 5%), talc (corresponding to 60% of polymer), and an appropriate amount of deionized water, and then coating in the fluidized bed.

(3) Preparation of composite sustained-release pellets: accurately weighing a certain amount of sitagliptin, dissolving it in deionized water, and spraying the aqueous solution of sitagliptin to the surfaces of the sustained-release pellets of fructose-1,6-bisphosphate using a fluidized bed device, so as to obtain the composite pellets.

The inventors believe that because of the respective pharmacokinetic characteristics of fructose-1,6-bisphosphate and sitagliptin and the special mechanism of the combination thereof, the research of the preparation forms focuses on a simultaneous or sequential release of these two medicaments. Therefore, common preparation forms including these two medicaments, or sustained-controlled release preparations, including composite sustained-release preparations including sustained-release pellets, double-layer matrix tablets, and film-controlled release tablets, all have good development and application prospects.

It can be understood by those skilled in the art that the present invention is also suitable for clinically combining separate preparations of FBP and stabilizers, which are simultaneously or sequentially administrated according to the actual situation, preferably the stabilizer is administrated 30 minutes in advance.

The present disclosure has the following main points and beneficial effects:

The exogenous fructose-1,6-bisphosphate (FBP) enters the body and is rapidly degraded, and such a rapid degradation is exacerbated with the extension of the treatment time, especially, the phenomenon of degradation becomes more serious with an increase in dosage. In this regard, it is difficult for the existing FBP preparations to produce and maintain an effective blood concentration, which greatly limits the medicinal application values of FBP, especially for the prevention and treatment of chronic diseases. The present invention first discloses that the gluconeogenesis pathway is involved in the rapid degradation of the exogenous FBP, particularly activation of this metabolic pathway leads to the gradual disappearance of the efficacy with prolonged treatment duration. Thereafter, it is found that the hypoglycemic agents can inhibit the rapid in vivo degradation of the exogenous FBP and greatly improve the overall efficacy of FBP, including anticancer efficacy. The research results support the use of a composite constituted by the hypoglycemic agents, as a stabilizer of FBP in the body and FBP in manufacturing the medicament for the prevention and treatment of metabolic diseases and metabolism-related diseases.

The inventiveness and scientific nature of the present invention are also reflected in the following aspects: as for the common pathological mechanism involved in a variety of different diseases (including neurodegenerative diseases, neurological disorders, obesity, diabetes, and tumors), i.e., the metabolic dysfunctions in which the metabolic disorders of differentiated cells include intensified glycolytic activity, weakened mitochondrial oxidative phosphorylation and associated oxidative damages, metabolic reprogramming of tumor cells including intensified glycolytic activity and de novo biosynthesis as well as weakened mitochondrial oxidative phosphorylation), the pharmacological effects of FBP on inhibiting excessive glycolysis and promoting mitochondrial oxidative phosphorylation are utilized for the treatment of various diseases.

Beneficial effects: a FBP composite preparation, with fructose-1,6-bisphosphate (FBP) and a blood concentration stabilizer thereof as main ingredients, is provided, and such a medicament has multiple advantages over the existing FBP preparations in which FBP is the only active component. First, the crucial problem that the medicinal applications of the existing FBP preparations are limited, i.e., the rapid in vivo degradation of the exogenous fructose-1,6-bisphosphate (FBP). In this way, the FBP composite preparation according to the present disclosure can produce a higher peak of the FBP blood concentration and a more stable blood concentration, thereby having more significant efficacy, and the FBP composite preparation can also reduce the dosage of FBP and reduce the toxicity caused by a large amount of inorganic phosphorus entering the systemic circulation after the hydrolysis of a large amount of FBP. In particular, the FBP composite preparation overcomes the problem of the existing FBP preparations that the in vivo metabolism of FBP is continuously accelerated with the extension of the treatment duration, and thus the FBP composite preparation has significant advantages in the treatment of various metabolic chronic diseases and metabolism-related chronic diseases. In addition, the stabilizer in the FBP composite preparation can improve the metabolic state through a mechanism different from the mechanism of FBP, such that the two can produce synergistic pharmacological effects mediated by different mechanisms. By selecting appropriate excipients, ratios of excipients and preparation methods, sustained and controlled release preparations, targeting nano-preparations, and preparations of different content specifications can be prepared, which improves clinical compliance of the medicament composition.

### BRIEF DESCRIPTION OF DRAWINGS

FIG. 1 illustrates a regulating effect of fructose-1,6-bisphosphate on metabolism in normal human astrocytes. FBP: fructose-1,6-bisphosphate. Notes: experimental data were analyzed by using one-way ANOVA, and significant differences between groups were detected by using LSD method. Treatment group vs. control group ***P<0.001.

FIG. 2 illustrates an inhibition of glycolysis in glioma cells by fructose-1,6-bisphosphate. FBP: fructose-1,6-bisphosphate.

FIG. 3 illustrates that fructose 1,6-bisphosphate blocks glycolysis intermediates flowing to biosynthesis. FBP: fructose 1,6-bisphosphate; GAP: glyceraldehyde 3-phosphate; PEP: phosphoenolpyruvate; Pyr: pyruvate; G6P: glucose 6-phosphate; PGA: glyceryl triphosphate; La: lactic acid; Ser: serine; Gly: glycine; R5P: ribose 5-phosphate; ATP: adenosine triphosphate; UTP: uridine triphosphate; A: adenosine; C: cytidine; U: uridine; T: thymidine; A: adenine; G: Guanine. Notes: experimental data were analyzed by using one-way ANOVA, and significant differences between groups was detected by using LSD method. Treatment group vs. control group ***P<0.001.

FIG. 4 illustrates influences of repeated treatment with fructose-1,6-bisphosphate and repeated treatment with a combination of fructose-1,6-bisphosphate and metformin or sitagliptin on a protein level of fructose-1,6-biphosphosidase 1, FBP: fructose-1,6-bisphosphate; FBPase1: fructose 1,6-biphosphosidase 1; Met: metformin; STG: sitagliptin.

FIG. 5 illustrates effects of metformin, sitagliptin and insulin on peak concentration increase of fructose 1,6-bisphosphate and stabilization of blood concentration of FBP, FBP: fructose 1,6-bisphosphate; Met: metformin; STG: sitagliptin; Ins: insulin. Notes: Experimental data were analyzed using a least significant difference method. ***P<0.001, *P<0.05 vs. 0 hours (before administration), # P<0.05 vs. FBP group

### DESCRIPTION OF EMBODIMENTS

The present disclosure is further described with reference to the drawings and specific embodiments. However, it should be understood that the scope of the present invention is not limited to the following examples, the above implementations are all included in the scope of the invention, and any replacement based on the contents of the present invention shall fall within the protection scope of the present invention.

### Example 1. Regulation of Fructose-1,6-Bisphosphate on Metabolism in Normal Human Astrocytes

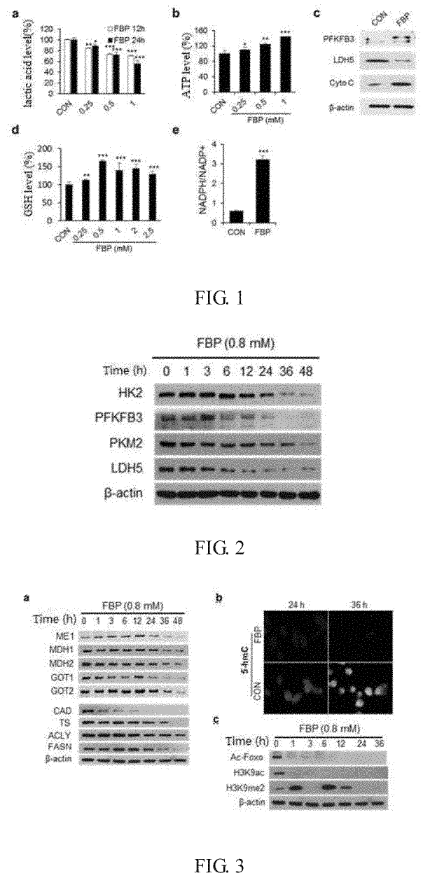

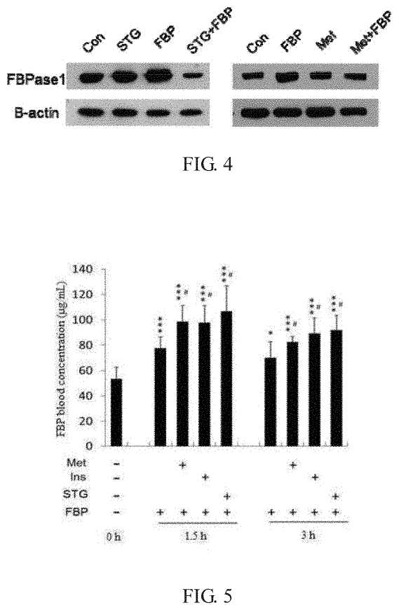

Normal human astrocytes (HA) were incubated in culture media containing different concentrations of trisodium fructose-1,6-bisphosphate salt (0 mM, 0.25 mM, 0.5 mM, 1 mM), and lactic acid level in the culture media and intracellular ATP level were measured after 12 h and 24 h. The results indicated that, compared with a control group, the lactic acid levels in the treatment groups at 12 h and 24 h decreased significantly with an increase in the concentration of trisodium fructose-1,6-bisphosphate salt (treatment group vs. control group: ***P<0.001); and the ATP levels at 24 h in the treatment groups increased significantly with the increase in the concentration of trisodium fructose-1,6-bisphosphate salt (treatment group vs. control group: ***P<0.001) (FIG. 1a, b).

The normal human astrocytes (HA) were incubated in a culture medium containing 0.8 mM of trisodium fructose-1,6-bisphosphate, and after 36 h, intracellular protein levels (β-actin as internal reference) of 6-Phosphofructo-2-kinase/fructose-2,6-bisphosphatase isoform 3 (PFKFB3), lactate dehydrogenase (LDH5), and cytochrome C (Cyto C) were measured by Western Blot. The experimental results indicated that, compared with the control group, the protein levels of PFKFB3 and LDH5 in the treatment groups were significantly reduced, while the protein level of Cyto C was significantly increased. (FIG. 1c)

The normal human astrocytes (HA) were incubated in culture media containing different concentrations of trisodium fructose-1,6-bisphosphate salt (0 mM, 0.25 mM, 0.5 mM, 1 mM, 2 mM, 2.5 mM), and after 36 h, reduced glutathione GSH level and a ratio of NADPH/NADP+ in the group of 1.6 mM trisodium fructose-1,6-bisphosphate in the cells were measured. The experimental results indicated that, compared with the control group, the GSH levels in the cells of the treatment groups were significantly increased (treatment group vs. control group: ***P<0.001), and the ratio of NADPH/NADP+ was significantly increased (treatment group vs. control group: ***P<0.001). (FIG. 1d, e)

The above experimental results indicated that the trisodium fructose-1,6-bisphosphate salt has effects of inhibiting glycolysis of normal human astrocyte HA, promoting tricarboxylic acid circulation and oxidative phosphorylation, and improving resistance to oxidative stress.

### Discussion and Summary

Astrocytes are the most abundant and versatile cell types in the brain. Specifically, the astrocytes are extremely active in metabolic activities, their metabolic activities are closely related to various functions, such as providing metabolic support to neurons and maintaining neurotransmitter dynamic equilibrium and redox dynamic equilibrium, etc., and thus metabolism disorders of the astrocytes, such as excessive glycolysis and weakened mitochondrial oxidative phosphorylation, are closely related to neurodegeneration and degenerative diseases. FBP can inhibit the excessive glycolysis of astrocytes, so as to reduce the accumulation of lactic acid; FBP can promote the oxidative phosphorylation to increase the ATP level; and FBP can further increase the levels of endogenous antioxidants such as NADPH and glutathione (GSH), so as to improve the resistance to oxidative damage. Therefore, the above research results strongly support the medical use of FBP in the prevention and treatment of neurodegenerative diseases.

### Example 2. Exogenous Fructose-1,6-Bisphosphate Cannot be Consumed by Tumor Cells, but Glycolytic Intermediates Thereof are Significantly Increased

Rat glioma cell strains (C6), human glioma cell strains (U87-MG, U-251, SHG-44) and patient-derived glioma cells (tumor 1, tumor 3) were incubated in a culture medium containing 1.6 mM of trisodium fructose-1,6-bisphosphate, and after 36 h, levels of glycolytic intermediates including glucose-6-phosphate G6P, fructose-1,6-bisphosphate FBP, glyceraldehyde 3-phosphate (GAP), dihydroxyacetone phosphate (DHAP), and glycerol 3-phosphoglyceric acid PGA in respective cells were measured by using LC-MS/MS; and additionally, the levels of fructose-1,6-bisphosphate in each glioma cell strain at 12 h and 36 h were measured. The experimental results indicated that, compared with the control group, the levels of the intracellular glycolytic products FBP, GAP, and DHAP in the treatment group treated with fructose-1,6-bisphosphate were significantly increased (treatment group vs. control group: ***P<0.001) (Table 1a); and the concentrations of FBP in the culture medium of the treatment group treated with fructose-1,6-bisphosphate did not decrease significantly with treatment time (N.S. P>0.1) (Table 1b). The experimental results indicated that the tumor cells do not consume exogenous FBP, and a part of FBPs entering the cell may undergo a first-step degradation reaction and produce GAP and DHAP along the glycolysis pathway, and stop at this step, thereby resulting in the accumulation of GAP and DHAP. In addition, the increased intracellular F6P level caused by the exogenous FBP suggests that FBP may also be degraded by fructose-1,6-biphosphatase (FBPase) in tumor cells.

TABLE 1a
Increase folds of intracellular glycolytic intermediates
(compared with the control group)

| Cell strain | F6P | FBP | GAP | DHAP | PGA |
|---|---|---|---|---|---|
| U87-MG | 47.31 | 149.69 | 1222.86 | 944.4 | 3.03 |
| C6 | 19.89 | 92.22 | 132.85 | 66.04 | 2.2 |
| KNS-89 | 1.89 | 21.97 | 19.75 | 8.74 | 3.28 |
| SHG-44 | 3.12 | 10.74 | 21.03 | 12.48 | 2.04 |
| Tumor 1 | 2.02 | 8.27 | 7.22 | 4.9 | 1.13 |
| Tumor 3 | 14.12 | 22.94 | 76.22 | 55.58 | 1.86 |

TABLE 1b
Levels of FBP in the culture medium (mg/ml)

| | Cell strain | | | | | |
|---|---|---|---|---|---|---|
| Time | U87-MG | C6 | U-251 | SHG-44 | Tumor 1 | Tumor 3 |
| 12 h | 0.76 | 1.09 | 1.35 | 1.07 | 0.17 | 0.68 |
| 36 h | 0.71 | 0.96 | 1.25 | 0.72 | 0.18 | 0.61 |

G6P: glucose-6-phosphate; FBP: fructose-1,6-bisphosphate; GAP: glyceraldehyde 3-phosphate; DHAP: dihydroxyacetone phosphate; and PGA: 3-phosphoglyceric acid.

### Example 3. Fructose-1,6-Bisphosphate Inhibits Glycolysis of Glioma Cells

Human glioma cell strains (U87-MG, KNS-89, SHG-44) were respectively incubated in culture media containing 0.8 mM of trisodium fructose-1,6-bisphosphate salt, and 1.6 mM of trisodium fructose-1,6-bisphosphate salt, and 1.6 mM of 2-deoxyglucose, and the contents of lactic acid as the final glycolytic product released by the cells in the culture media were measured after 12 h, 24 h, 36 h, and 48 h. The lactic acid level in the treatment group was significantly lower than that in the control group without addition of treatment agent (CON) (treatment group vs. control group: ***P<0.001) (Tables 2a-2c). Human glioma cell strain (U87-MG) was incubated in the culture medium containing 0.8 mM of trisodium fructose-1,6-bisphosphate salt for 1 h, 3 h, 6 h, 12 h, 24 h, 36 h, and 48 h, and level changes of key metabolic enzymes in the glycolytic pathway of cells were measured by Western Blot at the respective time points. It was found that hexokinase 2 (HK2), 6-phosphofructokinase 2 (PFKFB3), pyruvate kinase 2 (PKM2), and lactate dehydrogenase 5 (LDH5) were all quickly and continuously down-regulated (FIG. 2). The experimental results suggest that fructose 1,6-bisphosphate can inhibit the glycolysis of various glioma cells.

TABLE 2a
Relative levels of lactic acid in glioma cells U87-MG treated with
fructose-1,6-bisphosphate (compared with the control group)

| | Time point | | |
|---|---|---|---|
| Group | 12 h | 24 h | 36 h |
| CON | 1.00 ± 0.13 | 1.00 ± 0.07 | 1.00 ± 0.16 |
| 2-DG | 0.48 ± 0.16*** | 0.31 ± 0.07*** | 0.65 ± 0.10*** |
| FBP 0.8 mM | 0.96 ± 0.13*** | 0.73 ± 0.05*** | 0.56 ± 0.18*** |
| FBP 1.6 mM | 1.09 ± 0.11*** | 0.71 ± 0.05*** | 0.39 ± 0.03*** |

TABLE 2b
Relative levels of lactic acid in glioma cells KNS-89 treated with
fructose-1,6-bisphosphate (compared with the control group)

| | Time point | | |
|---|---|---|---|
| Group | 12 h | 24 h | 36 h |
| CON | 1.00 ± 0.10 | 1.00 ± 0.03 | 1.00 ± 0.07 |
| 2-DG | 0.31 ± 0.09*** | 0.27 ± 0.13*** | 0.31 ± 0.02*** |
| FBP 0.8 mM | 0.75 ± 0.11*** | 0.94 ± 0.16 | 0.78 ± 0.09*** |
| FBP 1.6 mM | 0.87 ± 0.03*** | 0.83 ± 0.20 | 0.62 ± 0.10*** |

TABLE 2c
Relative levels of lactic acid in glioma cells SHG-44 treated with
fructose-1,6-bisphosphate (compared with the control group)

| | Time point | | |
|---|---|---|---|
| Group | 12 h | 24 h | 36 h |
| CON | 1.00 ± 0.10 | 1.00 ± 0.0 | 1.00 ± 0.10 |
| 2-DG | 0.76 ± 0.15*** | 0.62 ± 0.13*** | 0.57 ± 0.04*** |
| FBP 0.8 mM | 1.06 ± 0.05 | 0.87 ± 0.01* | 0.83 ± 0.05*** |
| FBP 1.6 mM | 0.89 ± 0.09*** | 0.75 ± 0.07*** | 0.58 ± 0.06*** |

Notes:
the experimental data were analyzed by using one-way ANOVA, and significant difference between groups was detected by using LSD method.
(treatment group vs. control group
*P < 0.1: significant difference,
***P < 0.001: extremely significant difference).
FBP: fructose-1,6-bisphosphate; 2-DG: 2-deoxy-D-glucose.

### Example 4. Fructose 1,6-Bisphosphate Promotes Mitochondrial Oxidative Phosphorylation in Glioma Cells

Rat glioma cell strain (C6) and human glioma cell strain (KNS-89, SHG-44) were respectively incubated in media containing 0.8 mM of trisodium fructose-1,6-bisphosphate or 1.6 mM of trisodium fructose-1,6-bisphosphate, and after 36 h, it was observed that a ratio of ATP/ADP in each cell strain (the fructose 1,6-bisphosphate group vs. the control group ***P<0.001), and a ratio of NADH/NAD+ increased significantly (the fructose 1,6-bisphosphate group vs. the control group ***P<0.001), and in the meantime, the ATP level significantly increased (the fructose 1,6-bisphosphate group vs. the control group ***P<0.001) (Tables 3a-3b). The experimental results indicate that fructose 1,6-bisphosphate promotes the mitochondrial oxidative phosphorylation in the glioma cells.

TABLE 3a
Fructose-1,6-bisphosphate increases the
ratio of ATP/ADP in the glioma cells

Group

| Cell strain | CON | FBP |
|---|---|---|
| C6 | 1.00 ± 0.12 | 10.89 ± 1.37*** |
| KNS-89 | 1.00 ± 0.12 | 2.91 ± 0.03*** |
| SHG-44 | 1.00 ± 0.09 | 3.82 ± 0.68*** |

TABLE 3b
Fructose-1,6-bisphosphate increases the
ratio of NADH/NAD+ in the glioma cells

Group

| Cell stain | CON | FBP |
|---|---|---|
| C6 | 1.00 ± 0.12 | 7.76 ± 0.59*** |
| KNS-89 | 1.00 ± 0.06 | 9.44 ± 0.75*** |
| SHG-44 | 1.00 ± 0.03 | 3.40 ± 0.15*** |

Notes: the experimental data were analyzed by using one-way ANOVA, and significant difference between groups was detected by using LSD method. Treatment group vs. control group ***P<0.001 extremely significant difference. FBP: trisodium fructose-1,6-bisphosphate salt.

### Example 5. Trisodium Fructose 1,6-Bisphosphate Salt Blocks Glycolytic Intermediates Flowing to Biosynthesis

Human glioma cell strain (U87MG) was incubated in a medium containing ¹³C-labeled glucose (U-¹³C-Glc) and treated with 1.6 mM of trisodium fructose-1,6-bisphosphate for 36 h, and then, intermediate products of intracellular glycolysis pathway, pentose phosphate pathway, “one-carbon unit” metabolic pathway, and nucleic acid de novo synthesis pathway were measured using liquid-mass spectrometry (LC-MS/MS). The experimental results indicate that: (1) levels of glycolytic intermediates fructose 1,6-bisphosphate (FBP), glyceraldehyde 3-phosphate (GAP), and phosphoenolpyruvate (PEP) in the treatment group were significantly increased compared with those in the control group (vs. the control group ***P<0.001), and the level of the glycolytic product lactic acid (Lac) was significantly reduced (vs. the control group, ***P<0.001) (Table 4a); (2) serine (Ser) (M+3) produced by U-¹³C-Glc through a serine biosynthesis pathway in the treatment group was significantly increased (the control group 1±0.03, the treatment group 1.57±0.04, the treatment group vs. the control group ***P<0.001), and glycine (Gly) (M+2) produced by serine through the “one-carbon unit” metabolic pathway was significantly reduced (the control group 1±0.07, the treatment group 0.63±0.06, the treatment group vs. the control group ***P<0.001); (3) a proportion of the ¹³C-labeled 5-phosphate ribose (R5P) among the product 5-phosphate ribose of the pentose phosphate pathway decreased from 68.96±5.03% of the control group to 17.32±1.23% of the treatment group (the control group vs. the treatment group ***P<0.001); and (4) proportions of labeled ribose in free nucleic acid biosynthesis intermediates adenosine triphosphate (ATP), uridine triphosphate (UTP), adenosine (A), cytidine (C), uridine (U), and thymidine (T) significantly decreased compared with the untreated group (vs. the control group *P<0.05, ***P<0.001) (Table 4b).

The experimental results indicate that the trisodium fructose-1,6-bisphosphate can accumulate the glycolytic intermediates in the glycolytic pathway, reduce the glycolytic intermediates undergoing the pentose phosphate pathway, serine biosynthesis, and “one-carbon unit” metabolism, and in turn reduce de novo synthesis of nucleic acids.

TABLE 4a
Relative levels of glycolytic intermediates after the treatment
with fructose-1,6-bisphosphate (compared with the control group)

| | F6P | FBP | GAP | PEP | Lac |
|---|---|---|---|---|---|
| CON | 1 ± 0.01 | 1 ± 0.05 | 1 ± 0.18 | 1 ± 0.06 | 1 ± 0.01 |
| FBP | 2.06 ± 0.04*** | 5.01 ± 0.34*** | 4.27 ± 0.07*** | 8.00 ± 0.32*** | 1.06 ± 0.02 ^{N.S.} |

TABLE 4b
Proportions of ¹³C-labeled ribose in free nucleosides and nucleotides
after the treatment with fructose-1,6-bisphosphate

| | F6P | FBP | GAP | PEP | Lac |
|---|---|---|---|---|---|
| CON | 13.03 ± 0.38 | 41.00 ± 4.28 | 31.97 ± 1.11 | 59.40 ± 1.11 | 61.60 ± 1.95 |
| FBP | 2.79 ± 0.14*** | 34.99 ± 1.63* | 11.31 ± 1.75*** | 13.71 ± 0.54*** | 30.70 ± 2.11*** |

Notes: the experimental data were analyzed by using one-way ANOVA, and significant difference between groups was detected by using LSD method. Treatment group vs. control group (N.S.: no significant difference; *P<0.05 significant difference; ***P<0.001 extremely significant difference). F6P: fructose 6-phosphate; FBP: fructose-1,6-bisphosphate; GAP: glyceraldehyde 3-phosphate; PEP: phosphoenolpyruvate; Lac: lactic acid; ATP: adenosine triphosphate; UTP: uridine triphosphate; A: adenosine; C: cytidine; U: uridine.

### Example 6. Trisodium Fructose-1,6-Bisphosphate Blocks Tricarboxylic Acid Circulation Intermediate Flowing to Biosynthesis

Human glioma cell strain (U87MG) was incubated in a medium containing ¹³C-labeled glucose (U-¹³C-Glc) and treated with 1.6 mM of trisodium fructose-1,6-bisphosphate for 36 h, and then using liquid-mass spectrometry (LC-MS/MS), intracellular tricarboxylic acid circulation intermediates, tricarboxylic acid circulation intermediates-derived amino acids and nucleotide de novo synthesis pathway intermediates were measured. The experimental results indicate that: (1) levels of tricarboxylic acid circulation intermediates α-ketoglutarate (α-KG) and oxaloacetic acid (OAA) in the treatment group were significantly increased compared with the control group (vs. the control group ***P<0.001) (Table 5a); (2) levels of tricarboxylic acid circulation intermediates aspartic acid (Asp) and glutamic acid (Glu) in the treatment group were significantly reduced compared with the control group (vs. the control group ***P<0.001) (Table 5b); and (3) proportions of ¹³C-labeled purine ring and pyrimidine ring in free nucleosides and nucleotides in the treatment group significantly decreased (vs. the control group, **P<0.005, ***P<0.001) (Table 5c). The experimental results indicate that trisodium fructose-1,6-bisphosphate can block the conversion of tricarboxylic acid circulation intermediates into amino acids, thereby blocking the involved de novo synthesis of nucleic acids.

TABLE 5a
Relative levels of some tricarboxylic acid circulation
intermediates after the treatment with fructose-1,6-
bisphosphate (compared with the control group)

| | α-KG | OAA |
|---|---|---|
| CON | 1 ± 0.01 | 1 ± 0.14 |
| FBP | 1.61 ± 0.01*** | 1.87 ± 0.04*** |

TABLE 5b
Relative levels of some amino acids after the treatment with
fructose-1,6-bisphosphate (compared with the control group)

| | Asp | Glu |
|---|---|---|
| CON | 1 ± 0.02 | 1 ± 0.05 |
| FBP | 0.16 ± 0.07*** | 0.35 ± 0.04*** |

TABLE 5c
Proportions of ¹³C-labeled purine ring and pyrimidine ring in free nucleosides
and nucleotides after the treatment with fructose-1,6-bisphosphate

| | ATP | A | G | UTP | U |
|---|---|---|---|---|---|
| CON | 38.26 ± 1.50 | 12.41 ± 1.19 | 53.75 ± 1.47 | 50.67 ± 0.96 | 20.58 ± 2.30 |
| FBP | 23.77 ± 0.57*** | 7.70 ± 1.50** | 41.83 ± 4.06** | 18.19 ± 0.87*** | 8.33 ± 0.90*** |

Notes:
the experimental data were analyzed by using one-way ANOVA, and significant difference between groups was detected by using LSD method.
Treatment group vs. control group
(**significant difference;
***P < 0.001: extremely significant difference).
FBP: fructose-1,6-bisphosphate; α-KG: α-ketoglutarate; OAA: oxaloacetic acid; Asp: aspartic acid; Glu: glutamic acid; ATP: adenosine triphosphate; UTP: uridine triphosphate; A: adenosine; C: cytidine; U: uridine.

### Example 7. Trisodium Fructose-1,6-Bisphosphate Blocks Tricarboxylic Acid Intermediates Flowing Out of Mitochondria, Destroys the Epigenetic Characteristics of Tumors, and Widely Down-Regulates Protein Levels of Tumor Metabolic Enzymes

Human glioma cell strain (U87MG) was cultured in a medium containing 1.6 mM of trisodium fructose-1,6-bisphosphate for 36 h, and then cytoplasm and mitochondria were separated. Levels of tricarboxylic acid circulation intermediates in cytoplasm and mitochondria were respectively measured by LC-MS/MS. It was found that, the levels of the tricarboxylic acid circulation intermediates acetyl-CoA (Ac-CoA), citric acid (Cit), α-ketoglutarate (α-KG), and oxaloacetic acid (OAA) in cytoplasm were significantly reduced in the treatment group (vs. the control group **P<0.01; ***P<0.001); those levels in mitochondria were significantly increased (vs. the control group ***P<0.001) (see Table 6); and in the meantime, protein levels of malate shuttle-related enzymes (ME1, MDH1, MDH2, GOT1, GOT2) between cytoplasm and mitochondria were decreased significantly over time (FIG. 3a). The experimental results show that the trisodium fructose 1,6-bisphosphate can block the tricarboxylic acid circulation metabolism intermediates flowing out of the mitochondria.

Human glioma cell strain (U87MG) was respectively cultured in the medium containing 1.6 mM of trisodium fructose-1,6-bisphosphate for 0, 1, 3, 6, 12, 24, 36, and 48 h, and the changes of protein levels of enzymes related to fatty acid and nucleic acid biosynthetic pathway were investigated by Western Blot (WB). It was found that the protein levels of the enzymes related to fatty acid and nucleic acid biosynthesis (CAD, TS, ACL, FASN) in the treatment group were decreased significantly over time (FIG. 3b). The experimental results show that fructose 1,6-bisphosphate can widely down-regulate metabolic enzymes in tumor cells.

Human glioma cell strain (U87MG) was respectively cultured in the medium containing 1.6 mM of trisodium fructose-1,6-bisphosphate for 24 h and 36 h, and then a level of 5-hydroxymethylcytosine (5-hmC) was measured by an immunocytochemistry method. It was found that 5-hmC in the treatment group was significantly increased; and at the same time, the epigenetic related proteins of tumor cells (Ac-Foxo, H3K9ac, H3K9me2) were quickly down-regulated. The experimental results show that fructose 1,6-bisphosphate can change the epigenetic characteristics of tumor cells (FIG. 3c).

TABLE 6
Relative levels of tricarboxylic acid circulation intermediates
in cytoplasm and mitochondria (compared with the control group)

| | | Ac-CoA | Cit | α-KG | OAA |
|---|---|---|---|---|---|
| Cyto | CON | 1 ± 0.08 | 1 ± 0.04 | 1 ± 0.07 | 1 ± 0.12 |
| | FBP | 0.02 ± 0.01*** | 0.28 ± 0.01*** | 1.20 ± 0.12* | 0.19 ± 0.01*** |
| Mito | CON | 1 ± 0.04 | 1 ± 0.06 | 1 ± 0.02 | 1 ± 0.02 |
| | FBP | 1.38 ± 0.11*** | 1.02 ± 0.06 | 2.42 ± 0.04*** | 2.02 ± 0.13*** |

Notes:
the experimental data were analyzed by using one way ANOVA, and significant difference between groups was detected by using LSD method.
Treatment group vs. control group
(*P < 0.05: significant difference,
***P < 0.001: extremely significant difference).
FBP: trisodium fructose-1,6-bisphosphate; Ac-CoA: acetyl-CoA; Cit: citric acid; α-KG: α-ketoglutarate; OAA: oxaloacetic acid; Cyto: cytoplasm; Mito: mitochondria.

### Example 8. Trisodium Fructose-1,6-Bisphosphate Disrupts Redox Balance in Glioma Cells

Rat glioma cell strain (C6) and human glioma cell strain (KNS-89) were respectively cultured in a medium containing 0.8 mM of trisodium fructose-1,6-bisphosphate, and a level of intracellular reactive oxygen species (ROS) was gradually increased with the treatment time (Table 7a), while mitochondrial membrane potential (MMP) gradually decreased (Table 7b).

Rat glioma cell strain (C6) and human glioma cell strain (KNS-89, SHG-44) were respectively cultured in a medium containing 1.6 mM of trisodium fructose-1,6-bisphosphate for 36 h, and important antioxidants in cells were measured by liquid-mass spectrometry (LC-MS/MS). The experimental results show that the levels of glutathiones (GSH, GSSG) decreased sharply (Table 7c), and the ratio of NADPH/NADP+ also decreased sharply (Table 7d).

The experimental results reveal that trisodium fructose-1,6-bisphosphate increase generation of the reactive oxygen species, inhibit synthesis of the antioxidant glutathione, and prevent the conversion of NADP+ to NADPH, thereby disrupting the redox balance of glioma cells from multiple aspects.

TABLE 7a
Relative level of reactive oxygen species (compared with the control group)

| | 0 h | 12 h | 24 h | 48 h | 72 h |
|---|---|---|---|---|---|
| C6 | 1 ± 0.04 | 1.26 ± 0.04*** | 1.89 ± 0.03*** | / | / |
| U251 | 1 ± 0.02 | 1.20 ± 0.04 | 1.19 ± 0.03 | 1.42 ± 0.03*** | 1.82 ± 0.03*** |

TABLE 7b
Relative level of mitochondrial membrane potential
(compared with the control group)

| | 0 h | 12 h | 24 h | 48 h | 72 h |
|---|---|---|---|---|---|
| U251 | 1 ± 0.03 | 1.08 ± 0.03 | 1.18 ± 0.01 | 0.6 ± 0.06*** | 0.4 ± 0.07*** |
| C6 | 1 ± 0.08 | 0.8 ± 0.14*** | 0.6 ± 0.11*** | 0.1 ± 0.09*** | 0.1 ± 0.07*** |

TABLE 7c
Relative contents of GSH and GSSG
(compared with the control group)

| | | C6 | KNS-89 | SHG-44 |
|---|---|---|---|---|
| GSH | CON | 1 ± 0.01 | 1 ± 0.01 | 1 ± 0.01 |
| | FBP | 0.69 ± 0.01*** | 0.01 ± 0.00*** | 0.67 ± 0.01*** |
| GSSG | CON | 1 ± 0.01 | 1 ± 0.04 | 1 ± 0.01 |
| | FBP | 0.06 ± 0.00*** | 0.33 ± 0.02*** | 0.38 ± 0.01*** |

TABLE 7d
Relative ratio of NADPH to NADP+
(compared with the control group)

| | | C6 | KNS-89 | SHG-44 |
|---|---|---|---|---|
| NADPH/ | CON | 1 ± 0.11 | 1 ± 0.03 | 1 ± 0.02 |
| NADP+ | FBP | 0.14 ± 0.01*** | 0.44 ± 0.04*** | 0.39 ± 0.01*** |

Notes:
the experimental data were analyzed by using one way ANOVA, and significant difference between groups was detected by using LSD method.
Treatment group vs. control group
***P < 0.001: extremely significant difference.
FBP: trisodium fructose-1,6-bisphosphate; GSH: reduced glutathione; GSSG: oxidized glutathione

### Discussion and Summary (Example 3 to Example 8): FBP Reverses Metabolic Characteristics of Tumors

Tumor cells undergo metabolic reprogramming, and particularly, produce a large number of glycolytic intermediates and tricarboxylic acid circulation intermediates, and utilize these intermediates for biosynthesis, thereby providing prerequisites for rapid division, proliferation, and growth of tumor cells. In addition, acetyl-CoA, fumaric acid, and succinic acid, which are derived from the tricarboxylic acid circulation intermediate products, support the epigenetic characteristics of tumors, and thus participate in the regulation of oncogene protein expression up-regulation and cancer suppressor protein expression down-regulation. FBP can reverse the metabolic characteristics of tumors, destroy the tumor metabolism network, and thus has significant anticancer activities in vivo and in vitro. Outstanding performances include promoting the entry of glucose and glutamine into the tricarboxylic acid circulation and oxidative phosphorylation, as well as blocking the intermediates of the glycolysis and tricarboxylic acid circulation flowing to biosynthesis and blocking the tricarboxylic acid intermediates flowing out of mitochondria, thereby destroying the epigenetic characteristics of tumors and widely down-regulating the protein levels of tumor metabolic enzymes. The research results strongly support the medicinal use of FBP in the treatment of various tumors.

### Example 9. Long-Term Treatment with Trisodium Fructose-1,6-Bisphosphate Leads to a Stress Increase in Protein Levels of Fructose-1,6-Bisphosphatase and a Decrease in Blood Concentration of Fructose-1,6-Bisphosphate, and Metformin and Sitagliptin Phosphate can Resist Such Fructose-1,6-Bisphosphate Metabolic Changes

180-200 g SD rats were divided into 4 groups: a saline control group, a trisodium fructose-1,6-bisphosphate hydrate group (500 mg/kg, i.g), a metformin group (150 mg/kg, i.g) or a sitagliptin group (20 mg/kg, i.g), and a trisodium fructose-1,6-bisphosphate and metformin or sitagliptin combination group, 5 rats in each group. All groups were intragastrically treated, metformin or sitagliptin was administrated 0.5 h before fructose-1,6-bisphosphate, and this was repeated in 14 consecutive days. At 3 hours after the last intragastric treatment with fructose-1,6-bisphosphate, rat blood was collected to detect the blood concentration of FBP, kidney tissue was taken and the protein level of fructose-1,6-bisphosphatase 1 was measured by Western Blot. The results indicate that: compared with the control group, the protein level of fructose-1,6-bisphosphatase 1 in the fructose-1,6-bisphosphate group was significantly up-regulated after the long-term treatment, the metformin or sitagliptin group was not significantly changed, and the metformin or sitagliptin combination group returned to normal levels; correspondingly, the blood concentration of fructose-1,6-bisphosphate in the fructose-1,6-bisphosphate group could not be maintained, and it was reduced to 60 μg/ml at 3 hours after the treatment, being the same as the normal FBP level in the body; the blood concentration thereof was significantly increased when metformin was combined, and reached 99.23 μg/ml at 3 hours after treatment, compared with the control group **P<0.01; compared with the metformin group, *P<0.05 (Table 4). Therefore, the long-term treatment with fructose-1,6-bisphosphate may cause a significant up-regulation of the protein level of fructose-1,6-bisphosphatase 1 such that fructose-1,6-bisphosphate is more easily degraded in the body, and a high and stable blood concentration cannot be maintained, thereby affecting the antitumor efficacy of fructose-1,6-bisphosphate. Metformin and sitagliptin do not affect the normal expression of FBPase1 in tissues, and thus can effectively resist the stress increase in the fructose-1,6-bisphosphatase 1 caused by fructose-1,6-bisphosphate, and restores the fructose-1,6-bisphosphatase 1 to a normal level, which is conducive to the stabilization of the blood concentration of FBP in the body, thereby exerting stronger antitumor efficacy.

TABLE 4
Influence on blood concentration of fructose-1,6-bisphosphate by repeated treatment
with fructose-1,6-bisphosphate individually and in combination with metformin

| | Con | Met | FBP | Met + FBP |
|---|---|---|---|---|
| FBP blood | 62.10 ± 8.70 | 73.73 ± 15.50 | 60.63 ± 10.20 | 99.23 ± 28.90**^{;}* |
| concentration | | | | |
| (μg/ml) | | | | |

Notes:
the experimental data were analyzed by using one way ANOVA, and significant difference between groups was detected by using LSD method.
Met + FBP group vs. control group/FBP group:
**P < 0.01;
Met + FBP group vs. Met group:
*P < 0.05.
FBP: fructose-1,6-bisphosphate; Met: metformin.

### Example 10. Stabilizing Effects of Metformin, Sitagliptin and Insulin on Blood Concentration of Fructose-1,6-Bisphosphate

Six-week-old ICR mice were divided into 4 groups: a saline control group, a trisodium fructose-1,6-bisphosphate hydrate group (500 mg/kg, i.g), a trisodium fructose-1,6-bisphosphate and metformin (150 mg/kg, i.g) combination group, a trisodium fructose-1,6-bisphosphate and sitagliptin (20 mg/kg) combination group, and a trisodium fructose-1,6-bisphosphate and insulin (4 U/kg) combination group, 7 mice in each group. All groups were intragastrically treated, metformin, sitagliptin and insulin were administrated 0.5 h before fructose-1,6-bisphosphate. 1.5 h and 3 h after the treatment with fructose 1,6-bisphosphate, the blood of mice in each group was collected to separate plasma, and the fructose 1,6-bisphosphate concentration in plasma was measured by the enzyme method. The results indicate that, in the fructose 1,6-bisphosphate group, FBP concentration in plasma was increased from 53.3 μg/ml to 77.5 μg/ml at 1.5 hours after the treatment, and was decreased to 70 μg/ml after 3 hours (the fructose 1,6-bisphosphate group, 1.5 hour vs. 0 hour *P<0.001); when combined with metformin, insulin, or sitagliptin, FBP blood concentration was increased to 97.5 μg/ml, 97.5 μg/ml, and 106 μg/ml after 1.5 hours, and the blood concentration of fructose-1,6-bisphosphate in each group after 3 hours were 82.5 μg/ml, 89.2 μg/ml, and 91.7 μg/ml, respectively, which were higher than those in the control group (in the combination group, 1.5 h, 3 h vs. 0 h *P<0.001), and compared with the fructose-1,6-bisphosphate group, a peak concentration and a maintenance time of the blood concentration were significantly increased in the combination group (1.5 hours, the combination group vs. the fructose-1,6-bisphosphate group ^{#} P<0.05, and 3 hours, the sitagliptin combination group and the insulin combination group vs. the FBP group ^{#} P<0.05). The above results indicate that the combination of FBP with metformin, sitagliptin or insulin can effectively increase the peak concentration of FBP and the maintenance time of the blood concentration of FBP in the body, thereby effectively improving the in vivo antitumor effect of FBP.

### Discussion and Summary (Example 9 and Example 10): Hypoglycemic Agents can Increase and Maintain FBP Blood Concentration, Preventing the Acceleration of FBP Metabolism with Prolonged Treatment Time

A long-term treatment with FBP in tumor model animals may cause the up-regulation of the protein level of fructose-1,6-biphosphatase (FBPase1), a FBP-degrading enzyme, and accelerate the degradation of FBP, so that FBP blood concentration may be gradually decreased with the extension of treatment time, thereby hindering the exertion of anti-cancer effects of FBP. In this regard, the present invention further explores the stabilizing effects of hypoglycemic agents including metformin, sitagliptin, and insulin on FBP blood concentration, and seeks to overcome the shortcomings of FBP being rapidly degraded in the body, which is not conducive to the exertion of its anticancer effect. The research results prove that these hypoglycemic agents can increase and maintain FBP blood concentration, and prevent the acceleration of FBP metabolism with the extension of the treatment time. Particularly, different hypoglycemic agents including metformin, sitagliptin, and insulin have different mechanisms, but all produce pharmacological effects of inhibiting gluconeogenesis. Therefore, based on the key role of the gluconeogenesis enzyme fructose-1,6-biphosphatase (FBPase1) in the degradation of the exogenous FBP, the above research results indicate that inhibitors of the fructose-1,6-biphosphosidase, as well as the existing hypoglycemic agents having different mechanisms and emerging noval hypoglycemic agents in the future all can inhibit the in vivo degradation of exogenous FBP, thereby improving the medicinal application of the exogenous FBP.

### Example 11. Metformin and Sitagliptin do not Significantly Affect the Effect of Trisodium Fructose-1,6-Bisphosphate on Human Intestinal Cancer Cells In Vitro

Human intestinal cancer cell strains SW620 and HCT-8, which had been incubated for 24 hours, were respectively incubated in a medium containing 0.8 mM of trisodium fructose-1,6-bisphosphate, a medium containing 0.2 mM of metformin/100 μM of sitagliptin, and a medium containing 0.8 mM of trisodium fructose-1,6-bisphosphate and 0.2 mM of metformin/100 μM of sitagliptin for 72 h. A control group (Con) was not treated with the agent. Cell viability was determined by Sulforhodamine B (SRB) staining analysis method. The results show that 41% of SW620 cell viability was inhibited by 0.8 mM of FBP, 0.2 mM of metformin did not affect the SW620 cell viability, and the combination of these two agents did not affect the efficacy of trisodium fructose-1,6-bisphosphate; 41% of HCT-8 cell viability was inhibited by 0.8 mM of trisodium fructose-1,6-bisphosphate, 100 μM of sitagliptin did not affect HCT-8 cell viability, and the combination of these two agents did not affect the efficacy of trisodium fructose-1,6-bisphosphate (the trisodium fructose-1,6-bisphosphate group vs. the control group, ***P<0.001; the combination group vs. the control group, ***P<0.001; the combination group vs. the trisodium fructose-1,6-bisphosphate group, no significant difference) (Table 5). The experimental results show that the in vitro experiments using trisodium fructose-1,6-bisphosphate in combination with metformin or sitagliptin have neither antagonistic effect nor obvious synergistic effect on the anti-intestinal cancer effect of the trisodium fructose-1,6-bisphosphate.

TABLE 5
Influence of fructose-1,6-bisphosphate in combination with metformin
or sitagliptin on human intestinal cancer cell viability

| | Con | Met | FBP | Met + FBP |
|---|---|---|---|---|
| SW620 | 100 ± 2.99 | 95.92 ± 7.54 | 49.90 ± 3.91*** | 42.67 ± 2.57*** |
| | Con | STG | FBP | STG + FBP |
| HCT-8 | 100 ± 2.57 | 95.43 ± 3.79 | 85.93 ± 3.29*** | 79.10 ± 6.17*** |

Notes:
the experimental data were analyzed by using one way ANOVA, and significant difference between groups was detected by using LSD method (vs. control group (Con),
***P < 0.001).
FBP: fructose-1,6-bisphosphate; Met: metformin; STG: sitagliptin.

### Example 12. Metformin and Sitagliptin have No Significant Influence on the In Vitro Anti-Human Liver Cancer Cell Effect of Trisodium Fructose-1,6-Bisphosphate

Human liver cancer cell strains Bel-7402 and huh-7, which had been incubated for 24 hours, were respectively incubated in a medium containing 1.6 mM or 0.8 mM of trisodium fructose-1,6-bisphosphate, a medium containing 0.2 mM of metformin or 25 μM of sitagliptin, and a medium containing 0.8 mM of trisodium fructose-1,6-bisphosphate and 0.2 mM of metformin/25 μM of sitagliptin for 72 h. A control group (Con) was not treated with the agent. Cell viability was determined by Sulforhodamine B (SRB) staining analysis method. The results show that 41% of Bel-7402 cell viability was inhibited by 0.8 mM of FBP, 0.2 mM of metformin inhibited 5% of the Bel-7402 cell viability, and the combination of these two agents did not affect the efficacy of trisodium fructose-1,6-bisphosphate; 20% of Huh-7 cell viability was inhibited by 0.8 mM of trisodium fructose-1,6-bisphosphate, there was no significant difference with respect to the cell viability between 25 μM of sitagliptin and the control group, and the combination of these two agents did not affect the efficacy of trisodium fructose-1,6-bisphosphate (the trisodium fructose-1,6-bisphosphate group vs. the control group, ***P<0.001; the combination group vs. the control group, ***P<0.001; the combination group vs. the trisodium fructose-1,6-bisphosphate group, no significant difference) (Table 6). The experimental results show that the in vitro experiments using trisodium fructose-1,6-bisphosphate in combination with metformin or sitagliptin have neither antagonistic effect nor obvious synergistic effect on the anti-liver cancer effect of the trisodium fructose-1,6-bisphosphate.

TABLE 6
Influence of fructose-1,6-bisphosphate in combination with metformin
or sitagliptin on human liver cancer cell viability

| | Con | Met | FBP | Met + FBP |
|---|---|---|---|---|
| Bel-7402 | 100 ± 4.81 | 91.69 ± 1.62 | 60.43 ± 4.66*** | 60.51 ± 3.87*** |

| | Con | STG | FBP | STG + FBP |
|---|---|---|---|---|
| Huh-7 | 100 ± 4.38 | 92.08 ± 1.35 | 80.38 ± 3.63*** | 79.52 ± 11.85*** |

Notes:
the experimental data were analyzed by using one way ANOVA, and significant difference between groups was detected by using LSD method (vs. control group (Con),
***P < 0.001).
FBP: fructose-1,6-bisphosphate; Met: metformin; STG: sitagliptin.

### Example 13. Metformin and Sitagliptin have No Significant Influence on the In Vitro Anti-Human Melanoma Cell Effect of Trisodium Fructose-1,6-Bisphosphate

Mouse melanoma B16 cells, which had been incubated for 24 hours, were incubated in a medium containing 0.8 mM of trisodium fructose-1,6-bisphosphate, a medium containing 20 μM of sitagliptin, and a medium containing 0.8 mM of trisodium fructose-1,6-bisphosphate and 20 μM of sitagliptin for 72 h. A control group (Con) was not treated with the agent. Cell viability was determined by Sulforhodamine B (SRB) staining analysis method. The results show that 22% of B16 cell viability was inhibited by 0.8 mM of trisodium fructose-1,6-bisphosphate, 20 μM of sitagliptin inhibited 16% of the B16 cell viability, and the combination of these two agents did not affect the efficacy of trisodium fructose-1,6-bisphosphate (the sitagliptin group vs. the control group, ***P<0.001; the trisodium fructose-1,6-bisphosphate group vs. the control group, ***P<0.001; the combination group vs. the control group, ***P<0.001; the combination group vs. the trisodium fructose-1,6-bisphosphate group, no significant difference) (Table 7). The experimental results show that the in vitro experiments using trisodium fructose-1,6-bisphosphate in combination with sitagliptin have neither antagonistic effect nor obvious synergistic effect on the anti-melanoma effect of the trisodium fructose-1,6-biphosphate.

TABLE 7
Influence of fructose-1,6-bisphosphate in combination
with sitagliptin on melanoma B16 cell viability

| Con | Met | FBP | Met + FBP |
|---|---|---|---|
| 100 ± 2.89 | 86.84 ± 3.84 | 77.67 ± 7.29*** | 70.38 ± 5.01***^{;#} |

Notes:
the experimental data were analyzed by using one way ANOVA, and significant difference between groups was detected by using LSD method (vs. control group (Con),
***P < 0.001;
vs. STG group,
^{#}P < 0.05).
FBP: fructose-1,6-bisphosphate; STG: sitagliptin.

### Example 14. Metformin and Sitagliptin Enhance the In Vivo Antitumor Efficacy of Trisodium Fructose-1,6-Bisphosphate

According to the conventional method, mouse liver cancer cells H22 were seeded under the right armpit skin of adult male ICR mice, and 24 hours after the inoculation, the mice were randomly divided into the following experimental groups: a saline control group, a trisodium fructose-,6-bisphosphate (FBP) group (500 mg/kg, i.g), a metformin (Met) group (150 mg/kg, i.g), and an agent combination (F+M) group (FBP 500 mg/kg+Met 150 mg/kg, i.g); or a saline control group, a trisodium fructose-1,6-bisphosphate (FBP) group (500 mg/kg, i.g), a sitagliptin (STG) group (20 mg/kg, i.g), an agent combination (FBP+STG) group (FBP 500 mg/kg+STG 20 mg/kg, i.g), 7 mice in each group. The treatment was performed three times per day, in which metformin or sitagliptin was administrated 0.5 h before trisodium fructose-1,6-bisphosphate. This is repeated for 7 days, and situations of the animals during the experiments were observed. The animals were sacrificed 24 hours after the last treatment, the tumor masses were taken out and weighed, and the average tumor weight in each group of animals was used as an efficacy index.

As shown in Table 8: trisodium fructose-1,6-bisphosphate inhibits 54.39% of tumor growth (the trisodium fructose-1,6-bisphosphate group vs. the control group, ***P<0.001); there was no significant difference between the average tumor weight of the metformin group and the average tumor weight of the control group; 46.12% of tumor growth in the sitagliptin group was inhibited (the sitagliptin group vs. the control group, ***P<0.001), sitagliptin did not show an antitumor effect in the cell experiments, but in the in vivo experiment sitagliptin showed a certain antitumor effect, indicating that sitagliptin may play an antitumor effect by stimulating the immunity of the body; when FBP was used in combination with metformin or sitagliptin, the overall antitumor effect was substantially improved, reaching the inhibition ratios of 74.2% and 75.3%, respectively (the combination group vs. the control group, ***P<0.001; the metformin and trisodium fructose-1,6-bisphosphate combination group vs. the metformin group, ^{###} P<0.001, and vs. the trisodium fructose-1,6-bisphosphate group, ^{#} P<0.05; the sitagliptin and trisodium fructose-1,6-bisphosphate combination group vs. the sitagliptin group, and vs. the trisodium fructose-1,6-bisphosphate group, ^{#} P<0.05).

TABLE 8
Pharmacological efficacy of fructose 1,6-bisphosphate in combination with metformin
or sitagliptin against tumor growth of mouse liver cancer H22 model

| Group | Con | Met | FBP | Met + FBP |
|---|---|---|---|---|
| Tumor weight (g) | 1.66 ± 0.29 | 1.46 ± 0.23 | 0.91 ± 0.23*** | 0.43 ± 0.20***^{;###; &} |

| Group | Con | STG | FBP | STG + FBP |
|---|---|---|---|---|
| Tumor weight (g) | 1.48 ± 0.34 | 0.78 ± 0.31** | 0.67 ± 0.28*** | 0.38 ± 0.14***^{;#; &} |

Notes:
the experimental data were analyzed by using one way ANOVA, and significant difference between groups was detected by using LSD method (vs. the control group (Con),
***P < 0.001,
**P < 0.01;
vs. the Met or STG group,
^{###}P < 0.001,
^{#}P < 0.05;
and vs. FBP group, P < 0.05).
FBP: fructose 1,6-bisphosphate; Met: metformin; STG: sitagliptin.

### Discussion and Summary (Example 11 to Example 14): Hypoglycemic Agents can Significantly Enhance the In Vivo Antitumor Efficacy of FBP, but have No Obvious Influence on the In Vitro Antitumor Efficacy

The present invention has found that the combination of FBP and metformin, sitagliptin or insulin in a dose for treating diabetes can increase and stabilize the blood concentration of FBP, thereby significantly improving the overall anticancer efficacy of FBP. In contrast, metformin in a higher dose exceeding the hypoglycemic dose itself has certain anti-cancer activity but cannot improve the anti-cancer effect of FBP, indicating that metformin enhances the anticancer effect of FBP by increasing and stabilizing FBP blood concentration, not by the direct anticancer efficacy of its own. Sitagliptin has no obvious anticancer activity in vitro at the same concentration of FBP, and but has a certain anticancer activity in the mouse liver cancer H22 model in the clinical hypoglycemic dose. This overall anticancer activity may be attributed to the regulation of the overall glycometabolism by sitagliptin. Therefore, the research results support the application of the combination of FBP and hypoglycemic agents, especially sitagliptin, in the preparation of noval anticancer agents.

### Example 15. Combination of Trisodium Fructose-1,6-Bisphosphate and Sitagliptin Resists Weight Gain and Fat Accumulation Caused by High-Fat Diet

40 ICR male mice (17-19 g/per mouse) were divided into 2 groups. 8 normal animals were fed with basic feed and purified water, the remaining 32 obese animals were fed with high-fat diet and purified water, in which the high-fat diet contains 45% basic feed including 18% crude protein, 4% fat, 8% fiber, 1.5% calcium, 8% amino acid; and 55% additives including 13% refined lard, 3% soybean oil, 8% sugar, and peanuts, soybeans, egg, bone powder, sesame, corn, buckwheat, salt, various vitamins); after 4 weeks of breeding, the obese animals had an obesity rate of 8% compared to normal animals, and were then treated in different groups. The animals of the normal group (Naive) were still fed with the basic feed, and the model animals were equally divided into 4 groups and were further fed with the high-fat diet. In addition, according to the amount of water the mice had consumed, a certain dose of the agent was dissolved in drinking water. The animals in the model group (Model) were fed with the purified water, the animals in the fructose-1,6-bisphosphate group were fed with water containing 0.18% of 8-molecule hydrate of sodium fructose-1,6-bisphosphate (equivalent to FBP 300 mg/kg), the animals in the sitagliptin group were fed with water containing 0.012% of sitagliptin phosphate salt (equivalent to STG 20 mg/kg), and the animals in the combination group were fed with water containing FBP 500 mg/kg+STG 20 mg/kg. The treatment lasted continuously for 6 weeks, the body weight, food intake and water intake were measured per week, the body weight and body length (CM) were measured on the last day of the last week, and a Lee's INDEX (Lee's INDEX=body weight (g)\hat{ }(1/3) * 1000/body length (cm)) was calculated to evaluate the state of animal obesity. Then, the animals were sacrificed by cervical vertebra dislocation and the mice were dissected, the epididymal fat pad was taken and weighed, and a fat coefficient was calculated to further investigate the degree of obesity of the mice. Oral glucose tolerance of the mice was tested one day before sacrifice. After 12 hours of fasting in mice, 10% glucose was intragastrically administered, and the blood glucose of the mice was tested at 0 min, 15 min, 30 min, 60 min, and 120 min after the intragastric administration to investigate the glycometabolism changes in obese mice.

The following research results were obtained:

1. Only the combination of sitagliptin and FBP can resist the weight gain caused by the high-fat diets. The weight of the normal animals showed a time-dependent increase, increasing from 36.57±2.79 g to 42.07±3.3 g in 6 weeks. However, the weight of the animals of high-fat diets was increased with a speed significantly higher than that of the normal animals, and the weight was as high as 47.89±3.34 g after 6 weeks, which was significantly different from the normal group (the model group vs. the normal group, ***P<0.001), which indicates that the weight of the ICR mice fed with such a high-fat diet can be significantly increased in 6 weeks. In the trisodium fructose-1,6-bisphosphate group and the sitagliptin group, the weights were 45.93±5.1 g and 46.78±6.1 g, respectively, which were not significantly different from the model group. After the animals were treated with sitagliptin in combination with trisodium fructose-1,6-bisphosphate, the weight gain was significantly slowed. After 6 weeks, the weight of the animals in the combination group was 40.98±3.65 g, which was significantly lower than that of the animals in the model group (the combination group vs. the model group, ***P<0.001).

2. FBP can effectively resist an increase in Lee's INDEX caused by high-fat feed, and sitagliptin cannot further enhance the efficacy of FBP. The Lee's INDEX of the mice in the model group is 353.28±9.64, which has a significant difference compared with the Lee's INDEX of the animals in the normal group (337.05±9.96) (**P<0.01), indicating that the mice in the model group were obese. The Lee's INDEX of the animals in the sitagliptin group was 346.34±9.36, which was not significantly different from that in the model group. The Lee's INDEX of the animals in the trisodium fructose-1,6-bisphosphate group (326.14±11.10) and the Lee's INDEX of the animals in the sitagliptin and trisodium fructose-1,6-bisphosphate combination group (338.29±9.48) were significantly lower than the Lee's INDEX of the animals in the model group (***P<0.001). However, the combination with sitagliptin failed to further enhance the efficacy of FBP.

3. FBP can significantly resist an increase of fat coefficient caused by high-fat diet, sitagliptin does not have such an effect, but the efficacy of FBP is better when combined with sitagliptin. The fat coefficient of obese mice in the model group was 3.96±0.83%, which was significantly higher than that in the normal group (1.58±0.68%) (the model group vs. the normal group, ***P<0.001). The fat coefficients of the animals in the treatment groups were all decreased, and the fat coefficient of the animals in the sitagliptin group was 3.28±1.14%, which was not significantly different from the model group. The fat coefficient of the animals in the trisodium fructose-1,6-bisphosphate group (2.81±0.81%) and the fat coefficient of the animals in the sitagliptin and trisodium fructose-1,6-bisphosphate combination group (2.50±0.98%) were significantly different from that in the model group (the trisodium fructose-1,6-bisphosphate group vs. the model group *P<0.05, the sitagliptin and trisodium fructose-1,6-bisphosphate combination group vs. the model group **P<0.01), of which the combination group has the best anti-obesity effect.

4. FBP, sitagliptin and the combination thereof do not affect normal diet and normal blood glucose level

The experimental mice initially had an adaptive intake of the high-fat diet. At the beginning, an intake amount was slightly lower than the normal diet, after one week, an intake amount of the mice in the high-fat diet group was 6.68 g/each mouse/day, which was not significantly different from that in the normal group (6.71 g/each mouse/day). An intake amounts of the treatment groups were slightly lower than that of the model group, specifically, 5.99 g/each mouse/day in the sitagliptin group, 6.46 g/each mouse/day in the trisodium fructose-1,6-bisphosphate group, and 6.37 g/each mouse/day in the sitagliptin and trisodium fructose-1,6-bisphosphate combination group, but the appetite of the mice was not significantly influenced.

The high-fat diet in the experiment did not induce changes in glucose tolerance in obese mice, and FBP, sitagliptin and the combination thereof did not affect the normal blood glucose levels of mice. After 12 h of fasting, the blood glucose levels of the respective groups were 4.50±0.66 mmol/L in the normal group, 4.09±1.06 mmol/L in the model group, 5.21±1.22 mmol/L in the sitagliptin group, 4.24±1.12 mmol/L in the trisodium fructose-1,6-bisphosphate group, and 4.66±1.60 mmol/L in the sitagliptin and trisodium fructose-1,6-bisphosphate combination group, and no significant difference was found between the groups. After gastric glucose administration, there was a sharp increase in blood glucose in mice. At 15 min after the administration, the blood glucose levels were 12.91±2.57 mmol/L in the normal group, 13.26±3.63 mmol/L in the model group, 13.74±4.27 mmol/L in the sitagliptin group, 14.28±2.23 mmol/L in the trisodium fructose-1,6-bisphosphate group, and 13.72-3.83 mmol/L in the sitagliptin and trisodium fructose-1,6-bisphosphate combination group. After 30 min, the blood glucose level of each group began to drop, and the blood glucose level of each group returned to the initial level at 120 min. It can be seen that FBP, sitagliptin and the combination thereof did not affect the normal blood glucose level and the glucose tolerance.

In summary, it is proved that FBP can promote fat metabolism, and sitagliptin can further enhance the efficacy of FBP regarding promoting fat metabolism, which supports the medicinal uses of FBP in combination with sitagliptin for weight loss as well as prevention and treatment of type 2 diabetes.

### Discussion and Summary-FBP in Combination with Sitaglptin Resists Weight Gain and Fat Accumulation Caused by High-Fat Diet, and Thus a Compound Preparation Prepared by FBP in Combination with Sitagliptin can be Used for Weight Loss and Prevention of Fatty Liver and Type 2 Diabetes

As obesity not only directly affects quality of life, but also indicates metabolic disorders and subsequent diabetes, an important measure to prevent diabetes is to prevent obesity. Regarding obesity caused by a high-fat diet, a combination of FBP and sitagliptin has a significant counteracting effect, FBP alone can reduce the degree of obesity but not reduce the weight, and sitagliptin alone has not shown any significant efficacy, which strongly supports the use of the combination of FBP and sitagliptin in the preparation of weight loss agents and drugs for prevention of fatty liver and type 2 diabetes.

### Example 16. Trisodium Fructose-1,6-Bisphosphate Resists Peripheral Neuralgia Induced by Tumor Chemotherapy Agent Paclitaxel

In this study, paclitaxel (2.8 mg/kg, 10 ml/kg) was injected intraperitoneally every other day, 4 times in total (days 1, 3, 5, 7) to induce peripheral neuralgia model in ICR female mice with a body weight of 20-24 g, and this model was used to observe the preventive effect of fructose-1,6-bisphosphate on the peripheral neuralgia caused by the chemotherapeutic agent. The paclitaxel antitumor agent is one of the first-line agents for human malignant tumors. Dose-limiting toxicity of paclitaxel mainly includes neurotoxicity and myelosuppression, the latter has been successfully overcome by using granulocyte colony-stimulating factor. However, the neurotoxicity, behaving as neuropathic pain, is still a worldwide problem, because the pain caused by this chemotherapy is not sensitive to any clinically used analgesics, and thus some patients have to reduce the dose until withdrawing the agent, which seriously affects the effect of the chemotherapy or even causes a failure of the chemotherapy. Some pains caused by the paclitaxel chemotherapy may not be eliminated immediately after the agent withdrawal, and often continues for months or even years, which seriously affects the life quality of tumor patients. In view of this, the peripheral neuralgia caused by paclitaxel is a representative pain after cancer treatment, and the animal pain model induced by paclitaxel is also representative.

A hot plate method was used to screen mice having relatively uniform heat-sensitive responses for experiments. 21 qualified mice were divided into a blank control group (saline group, ip), a paclitaxel model group, and a trisodium fructose-1,6-bisphosphate hydrate (400 mg/kg, 10 ml/kg, ig) preventive treatment group, 7 mice in each group. Fructose-1,6-bisphosphate was administrated once per day, and the mice was pre-treated with fructose-1,6-bisphosphate 2 h before the administration of paclitaxel. After paclitaxel withdrawal, trisodium fructose-1,6-bisphosphate was continuously administrated until the end of the experiment.

The heat-sensitive response of hind paws of the mice was measured with a hot plate experiment (52° C.±0.3) at 2 pm to 4 pm every day. The bilateral hind paws of the mice were placed on a hot plate of a hot plate instrument. When the mice felt pain caused by thermal stimuli, the animal would lick its hind paws or retract its hind paws. In this way, a latent period of licking or retracting the hind paws was recorded. A shorter latent period represents a lower pain threshold. An increase in the pain threshold of the paclitaxel animals indicates a counteracting effect against the neuropathic pain induced by the chemotherapeutic agent. After the paclitaxel injection finished, the fructose-1,6-bisphosphate was continuously administrated and the heat-sensitive responses of the animals in each group were measured. Experimental results indicate that fructose-1,6-bisphosphate has a significant inhibitory effect on peripheral neuralgia in mice induced by paclitaxel. As shown in the figure, before paclitaxel administration, the hind paw retraction latent periods of the three groups of animals were substantially the same; on day 7, day 9, day 11, and day 13 after the paclitaxel administration, the hind paw retraction latent period of the model group was significantly shorter than that of the blank control group, indicating that paclitaxel had induced significant peripheral neuralgia (**P<0.01, ***P<0.001); and on day 7 and any other time point later, the latent period of the fructose-1,6-bisphosphate group was significantly longer than that of the model group (**P<0.01, ***P<0.001). The above experimental results support the clinical value of fructose-1,6-bisphosphate in preventing and treating pain after cancer treatment, especially the peripheral neuralgia caused by chemotherapy agents.

### Discussion and Summary: FBP Resists the Peripheral Neuralgia Induced by Anticancer Agent Paclitaxel, but has a Narrow Effective Dose Range

Paclitaxel, one of the commonly used chemotherapeutic agents, is widely used in ovarian cancer, breast cancer, lung cancer, head and neck cancer, and other malignant tumors, but has severe toxic side effects, among which neurotoxicity is a main dose-limiting toxicity. Similar to the clinical neurotoxic side effect, an anticancer dose of paclitaxel can rapidly induce a mouse peripheral neuralgia model; and a simultaneous treatment with an appropriate dose of fructose-1,6-bisphosphate and paclitaxel can significantly resist the peripheral neuralgia in mice induced by paclitaxel. In particular, low-dose FBP (200 mg/kg) and high-dose FBP (400 mg/kg) show opposite pharmacodynamic changes. That is, the high-dose FBP shows resistance against the peripheral neuralgia in the early stage of modeling (the early stage of FBP treatment) shows a tendency of resisting the peripheral neuralgia, but with the extension of treatment duration, such a tendency of agent efficacy gradually disappears; and the low-dose FBP shows a change tendency that the agent efficacy gradually increases with the extension of treatment duration. The research results support the potential of FBP in the prevention and treatment of neurotoxic side effects of chemotherapy agents, particularly in combination with the commonly used chemotherapeutic agents (including taxanes, vinblastines, platinum and proteasome inhibitors), and the efficacy of FBP cannot be improved by increasing the dose of FBP. In summary, the up-regulation of the level of FBP-degrading enzyme FBPase with the extension of the treatment period, as described above, explains the phenomenon of ineffectiveness of the high-dose of FBP, and such a deficiency of FBP can be overcome by using a composite preparation prepared by anti-diabetic agents and FBP.

### Summary of Research Results (Example 1 to Example 16)

The novel FBP medicament according to the present disclosure is characterized in that medicinal ingredients thereof include fructose-1,6-bisphosphate (FBP) and one or more components capable of slowing down the in vivo degradation of FBP (also referred as to FBP blood concentration stabilizer). The stabilizer is used as a medicinal ingredient, and is characterized by achieving the efficacy by increasing and stabilizing the blood concentration of FBP, but addition or synergism of the efficacy derived from the metabolic regulation effect of the stabilizer itself with the efficacy of FBP is not excluded. Therefore, it could be understand that the novel FBP medicament according to the present disclosure is applicable to the prevention and treatment of obesity and peripheral neuralgia caused by chemotherapeutic agents for the treatment of cancer, and is also applicable to the disclosed various indications of FBP, such as adjuvant treatment for improving myocardial ischemia and viral myocarditis caused by angina pectoris of coronary heart disease, acute myocardial infarction, arrhythmia, and heart failure; improvement for cerebral hypoxic symptoms caused by cerebral infarction, cerebral hemorrhage, etc.; prevention and treatment of blood system cancer and various solid cancers (Chinese invention patent: ZL201110066413.6); prevention and treatment of diabetes complications (Chinese invention patent: CN00112023.9); and treatment of epilepsy (Chinese invention patent: ZL201310498212.2) and neurodegenerative diseases (Chinese invention patent: CN01107519.8). Since the essence of the efficacy of the novel FBP medicament according to the present disclosure is metabolism regulation, i.e., improving metabolic function and correcting abnormal metabolic states, it could be understood that the indications for the novel FBP medicament also include metabolic diseases and diseases closely related to metabolic disorders (for example, mental disorders such as schizophrenia, depression, etc.).

### Example 17. Preparation of Double-Layer Tablet of Fructose-1,6-Bisphosphate-Sitagliptin

TABLE 9
Formulation of double-layer tablet

| | | Formulation (gram) | |
|---|---|---|---|
| Category | Name | Layer A | Layer B |
| Active component | Trisodium | — | 500 |
| | fructose-1,6-bisphosphate | | |
| | hydrate | | |
| Active component | Sitagliptin phosphate | 150 | — |
| Filler | Starch | 250 | — |
| Filler | Microcrystalline cellulose | 300 | — |
| Filler | Pregelatinized starch | — | 350 |
| Disintegrant | Croscarmellose sodium | 80 | 200 |
| Lubricant | Magnesium stearate | 77 | 41 |
| Binder | 2% hypromellose | 60 | 60 |

The active ingredient, the filler, and the binder were mixed respectively according to the formulations of the layer A and the layer B, wet granulation was performed by using a wet granulator (I stirring; II shear, 5 minutes), the granulates were dried and modified in a drying box at 60° C.; according to the formulations, the granulates of the layer A and the layer B were respectively mixed with the disintergrant and lubricant in a mixer for 40 min, and then compressed by a double-layer tablet compressing machine to obtain the double-layer tablet of fructose-1,6-bisphosphate-sitagliptin. The obtained double-layer tablet has intact and smooth appearance, and a friability ≤0.9%, no significant difference of tablet weight, and disintegration time limit ≤7 minutes. Each of the obtained double-layer tablets is 0.5 g/tablet, each contains 0.125 g of trisodium fructose-1,6-bisphosphate. In clinical use, 20 tablets for each oral administration, three times a day.

TABLE 10a
Dissolution rate test

| | Time (min) | 0 | 5 | 10 | 30 | 45 | 60 | 90 | 120 |
|---|---|---|---|---|---|---|---|---|---|
| Dissolution rate | Ultra-pure | 0.00 | 3.45 | 15.26 | 31.51 | 43.34 | 66.74 | 84.80 | 95.82 |
| of trisodium | water | | | | | | | | |
| fructose-1,6- | 0.1 mole of | 0.00 | 4.06 | 14.31 | 29.18 | 55.75 | 70.93 | 87.61 | 97.19 |
| bisphosphate | hydrochloric | | | | | | | | |
| (%) | acid | | | | | | | | |
| | PBS (pH = 6.8) | 0.00 | 5.24 | 16.75 | 32.69 | 56.79 | 72.18 | 86.58 | 96.20 |

TABLE 10b
Dissolution rate test

| | Time (min) | 0 | 5 | 10 | 15 | 30 | 45 | 60 |
|---|---|---|---|---|---|---|---|---|
| Dissolution rate | Ultra-pure water | 0.00 | 75.95 | 89.15 | 96.49 | 99.34 | 101.13 | 100.47 |
| of sitagliptin | 0.1 mole of | 0.00 | 70.23 | 85.27 | 95.99 | 98.40 | 99.90 | 99.52 |
| phospahte (%) | hydrochloric acid | | | | | | | |
| | PBS (pH = 6.8) | 0.00 | 80.45 | 90.80 | 98.08 | 100.28 | 101.58 | 86.58 |

TABLE 11
Stability

| | | 1 | 2 | 3 | 4 | 5 | 6 |
|---|---|---|---|---|---|---|---|
| Name | Sample | months | months | months | months | months | months |
| trisodium | 1 | 98.96 | 100.56 | 96.77 | 103.23 | 98.38 | 98.98 |
| fructose-1,6- | 2 | 96.20 | 97.48 | 100.59 | 96.36 | 99.66 | 97.36 |
| bisphosphate (%) | 3 | 97.04 | 98.26 | 101.84 | 98.99 | 103.17 | 97.06 |
| Sitagliptin | 1 | 98.10 | 102.37 | 98.48 | 97.05 | 102.94 | 100.58 |
| phosphate (%) | 2 | 101.35 | 96.17 | 98.68 | 99.39 | 97.57 | 99.38 |
| | 3 | 94.98 | 99.19 | 100.18 | 101.85 | 98.00 | 97.74 |

### Example 18. Preparation of Composite Sustained-Release Pellets of Fructose-1,6-Bisphosphate-Sitagliptin

Preparation of blank pellets: the active components and excipients were weighed according to trisodium fructose-1,6-bisphosphate:microcrystalline cellulose:lactose=6:2.5:1.5, the excipients were mixed homogenously after being sieved, and a soft material was formed after addition of water, and was then subjected to extrusion-spheronization to obtain pellets of trisodium fructose-1,6-bisphosphate. The obtained pellets were dried at 50° C. for 6 h, and sieved through 18-24 mesh sieve to prepare the pellets for later use.

Sustained-release pellet coating: a coating solution was prepared with Eudragit Ne30d (polymer concentration: 5%), talc (corresponding to 60% of polymer), and an appropriate amount of deionized water, and then coating was performed in the fluidized bed.

Preparation of the composite sustained-release pellets: a certain amount of sitagliptin was accurately weighed, and dissolved in deionized water, and the aqueous solution of sitagliptin was sprayed to the surface of the sustained-release pellets of fructose-1,6-bisphosphate using a fluidized bed device, so as to obtain the composite pellets.

It can be understand that, the medicinal forms of FBP include prototype fructose-1,6-bisphosphate, and pharmaceutically acceptable salts of fructose-1,6-bisphosphate, and prodrugs or derivatives thereof, including, but not limited to, salts and hydrates formed by ammonium, sodium, potassium, calcium, magnesium, manganese, copper, methylamine, dimethylamine, trimethylamine, butyric acid, acetic acid, dichloroacetic acid, hydrochloric acid, hydrobromic acid, sulfuric acid, trifluoroacetic acid, citric acid or acid radical of maleic acid that forms a compound. The preferred medicinal form is 8-molecule hydrate of trisodium fructose-1,6-bisphosphate. The FBP stabilizer includes the existing hypoglycemic substances such as dipeptidyl peptidase-4 (DPP-4) inhibitors represented by sitagliptin, glucagon-like peptide 1 (GLP-1) receptor agonists, biguanides represented by metformin, insulins, glitazones, and fructose-1,6-bisphosphatase inhibitors. The medicinal forms of these stabilizers can be any existing medicinal forms, a prototype form, or pharmaceutically acceptable salts of a prodrug or derivative thereof; including, but not limited to, salts and hydrates formed by ammonium, sodium, potassium, calcium, magnesium, manganese, copper, methylamine, dimethylamine, trimethylamine, butyric acid, acetic acid, dichloroacetic acid, hydrochloric acid, hydrobromic acid, phosphoric acid, sulfuric acid, trifluoroacetic acid, citric acid or acid radical of maleic acid that can form a compound.

In practical applications, FBP and any one or more of the above FBP blood concentration stabilizers in an appropriate ratio may act as active components of a medicament. The active components and pharmaceutically acceptable excipients or carriers are used to prepare various regular pharmaceutical preparations (including oral and injection preparations), suppositories, films, and various novel preparations applying novel materials and novel techniques (including, but not limited to, controlled release double-layer tablets, controlled release nano-preparations, microcapsules, microspheres, enteric preparations and various long-acting preparations). Preferably, the novel FBP medicament is prepared into a double-layer tablet that can achieve sequential release or a long-acting sustained-release preparation having controlled and sustained release characteristics. The double-layer tablet that can achieve sequential release is technically characterized in that the stabilizer can be preferentially released for 15 minutes to 60 minutes, preferably 30 minutes. Since the purpose of stabilizing the FBP blood concentration can be achieved by combing one of the stabilizers with FBP, in practical applications, it is preferential to combine FBP with one type of stabilizer, preferably sitagliptin, in the preparation of the novel FBP medicament.

What is claimed is:

1. Use of a composition of a fructose-1,6-bisphosphate and a blood concentration stabilizer thereof in manufacturing a medicament for preventing and treating metabolic diseases and metabolic dysfunction related diseases, wherein the medicament further comprises a pharmaceutically acceptable excipient or carrier.

2. The use of a composition of a fructose-1,6-bisphosphate and a blood concentration stabilizer thereof in manufacturing a medicament for preventing and treating metabolic diseases and metabolic dysfunction related diseases according to claim 1, wherein the blood concentration stabilizer comprises dipeptidyl peptidase-4 (DPP-4) inhibitors such as sitagliptin, glucagon-like peptide 1 (GLP-1) receptor agonists, biguanides such as metformin, insulins, glitazones also known as thiazolidinediones, and fructose-1,6-bisphosphatase inhibitors.

3. The use of a composition of a fructose-1,6-bisphosphate and a blood concentration stabilizer thereof in manufacturing a medicament for preventing and treating metabolic diseases and metabolic dysfunction related diseases according to claim 1, wherein pharmaceutical forms of fructose-1,6-bisphosphate comprise prototype fructose-1,6-bisphosphate and pharmaceutically acceptable salts of fructose-1,6-bisphosphate, and prodrugs or derivatives thereof, comprising, but not limited to, salts and hydrates formed by ammonium, sodium, potassium, calcium, magnesium, manganese, copper, methylamine, dimethylamine, trimethylamine, butyric acid, acetic acid, dichloroacetic acid, hydrochloric acid, hydrobromic acid, sulfuric acid, trifluoroacetic acid, citric acid or acid radical of maleic acid which forms a compound.

4. The use of a composition of a fructose-1,6-bisphosphate and a blood concentration stabilizer thereof in manufacturing a medicament for preventing and treating metabolic diseases and metabolic dysfunction related diseases according to claim 1, wherein a medicinal form is 8-molecule hydrate of trisodium fructose-1,6-bisphosphate.

5. The use of a composition of a fructose-1,6-bisphosphate and a blood concentration stabilizer thereof in manufacturing a medicament for preventing and treating metabolic diseases and metabolic dysfunction related diseases according to claim 1, wherein the metabolic diseases and metabolic dysfunction related diseases comprise: myocardial ischemia and viral myocarditis caused by angina pectoris of coronary heart disease, acute myocardial infarction, arrhythmia, and heart failure; cerebral infarction; cerebral hypoxia caused by cerebral hemorrhage or the like; blood system cancer; various solid tumors; diabetes and complications thereof; fatty liver; epilepsy; neurodegenerative diseases; and psychobehavioral disorders.

6. The use of a composition of a fructose-1,6-bisphosphate and a blood concentration stabilizer thereof in manufacturing a medicament for preventing and treating metabolic diseases and metabolic dysfunction related diseases according to claim 2, wherein in the medicament, a mass ratio of 8-molecule hydrate of trisodium fructose-1,6-bisphosphate to metformin is 1:0.1 to 1:1; a mass ratio of 8-molecule hydrate of trisodium fructose-1,6-bisphosphate to sitagliptin is 1:0.001 to 1:0.5; and a mass ratio of 8-molecule hydrate of trisodium fructose-1,6-bisphosphate to insulin is 1:0.02 to 1:0.002.

7. The use according to claim 1, wherein the medicament is in a pharmaceutical preparation form selected from injections, common tablets, granules, capsules, double-layer tablets, controlled release double-layer tablets, sustained release tablets, single-chamber controlled release tablets, dispersible tablets, enteric-coated tablets, enteric-coated capsules, timed release tablets, controlled-sustained release capsules, sustained release pellets, capsules containing micro pellets or small tablets, or targeting preparation.
